SEQUENCE LISTING (11 sequences)

SEQ ID NO: 1 - DNA, 2939 bp, Homo sapiens
gacgccgcgctcggagccgcgagggaaccgccgcccgcatggccccggcccgccgccccgccggagcccgcctgctgctcgtctacgcgggcctgctggccgccgccgccgcgggcctggggtccccggagcctggggcgccctcgaggagccgcgcccgcagggagccgccgcccgggaacgagctgccccggggccccggggagagccgcgcggggccggccgctcgtccgccggagcccaccgctgagcgtgcacacagcgtcgacccccgggacgcctggatgctcttcgtcaggcagagtgacaagggtgtcaatggcaagaagaggagcaggggcaaggccaagaagctgaagttcggcttgccagggccccctgggcctcccggtccccagggccccccaggccccatcatcccacccgaggcgctgctgaaggagttccagctgctgctgaaaggtgcggtgcggcagcgggagcgcgcggagcccgaaccctgtacgtgtggccccgccgggccggtcgctgcgagcctcgccccggtctcggccaccgccggggaggacgacgacgacgtggtgggggacgtgctggcactgctggccgcgcccctggccccggggccgcgggcgccgcgcgtggaggccgctttcctctgccgcctgcgccgggacgcgttggtggagcggcgcgcgctgcacgagcttggcgtctactacctgcccgacgccgagggtgccttccgccgcggcccgggcctgaacttgaccagcggccagtacagggcgcccgtggctggcttctacgctctcgccgccacgctgcacgtggcgctcggggagccgccgaggagggggccgccgcgcccccgggaccacctgcgcctgctcatctgcatccagtcccggtgccagcgcaacgcctccctggaggccatcatgggcctggagagcagcagtgagctcttcaccatctctgtgaatggcgtcctgtacctgcagatggggcagtggacctccgtgttcttggacaacgccagcggctgctccctcacagtgcgcagtggctcccacttcagtgctgtcctcctgggcgtgtgagcggccaccacaggcccttcctctcaggggcaaatggagcacagatctagacaatgtgtggacagtgtcagagtagcagtggccacatggaggaggaggcccacccggaactctgcccacactggccactgcagttcagcccacagagccactgcaggcaggcctacggacgtgacacgcacgctggtggtcccggagccagggttgattcaggacaccatcttgggctcttatccaggaaagaaagagtcggcgtgcctgggggcacctgctagtctccagctgcaggccgactctttcctggcctgctcagcacctgcccagatggcctctgcgtctttcctgtgcccagccccaccttttccacctctcttcatgttctcatggagtgcaaagtgcaccagccagggcccctacctgggagagggtcagctgacgcagggctgagggggctgccacagggacgtacgctgtgtgttcttactgcttagaagggacggggtcactcaccactcccctggtctccatctgggctccttggtcttccctgcccctcccctaaccgtgttctacctgccagtggagctgagcactgcctagcctgggccagaggggcactggacaagggcctttgggggacagtaagtctgggcccagcttctagttctaatgtgtgcaagattaactcacaaattcacctcagaaggcctttccaaatgggagctccactcttcccctcctctgcaaatcttcacagccaaggccccttccaccctctccagaggtggatgagatcccttttcccctcccctctgaggtgctgactcactggtaggagccacccaccagaggaaagatctagaacgtctttacagattggagacagccgggcctgctgacccatcatccgaatagctgaagcagagtcttccaccaggggtgccagggcctggctgggtccagcggctctgggatgagcctcccaagcatctttcccacttgggtggccatgcggcgctgacattggacaggtggtggacgagagatggtcccgagaaagggtggtcttgggagggctggtcccaagcctgctgtgctcctgtggcagtgatggggcctggggatggggacggcagctctcatgaggacacacaggctgtgagcccgcagcctcctcaaatgtagcctcccacattttccccaaagtacaggactgtcccagagtaggtagtgaagaggacaaggccctcggcagcgacctccagggcctcctacctgctgaggaagagttaacccactgcctccccacacaacaggctacgaagaacctggtgcctcaggacctcctgggagccaagctggtctggcaagggcgctcaggcctgggagagaagggagcaatggccagtcaccttcaccttctaactaactagcctccggatgaggtggctgccaccaggcccgaatgatccccaggagcccagcttccaaaccccaacatcgaatcaaacatctccatccccaagtgcagtaacacacaaaaaccaaacactctgccctgggaaaggcctggtgcgattctcagtaggactcacacccaccctacctagaagtactgggctggcctgggtactgcatccgtgtgttttgataagggggtgatgtggccacgcccttatctagatttcactttgtatccactgggcacagatattctagagaacttatctttcactcttgtaaaagccacatatccacatctctttcatttttctcagtgtgttatgcagcaatttattaaagtatttattgtctaataaatactgccaagtggaa

SEQ ID NO: 2 - DNA, 2951 bp, Homo sapiens
gacgccgcgctcggagccgcgagggaaccgccgcccgcatggccccggcccgccgccccgccggagcccgcctgctgctcgtctacgcgggcctgctggccgccgccgccgcgggcctggggtccccggagcctggggcgccctcgaggagccgcgcccgcagggagccgccgcccgggaacgagctgccccggggccccggggagagccgcgcggggccggccgctcgtccgccggagcccaccgctgagcgtgcacacagcgtcgacccccgggacgcctggatgctcttcgtcaggcagagtgacaagggtgtcaatggcaagaagaggagcaggggcaaggccaagaagctgaaggttccctttcagttcggcttgccagggccccctgggcctcccggtccccagggccccccaggccccatcatcccacccgaggcgctgctgaaggagttccagctgctgctgaaaggtgcggtgcggcagcgggagcgcgcggagcccgaaccctgtacgtgtggccccgccgggccggtcgctgcgagcctcgccccggtctcggccaccgccggggaggacgacgacgacgtggtgggggacgtgctggcactgctggccgcgcccctggccccggggccgcgggcgccgcgcgtggaggccgctttcctctgccgcctgcgccgggacgcgttggtggagcggcgcgcgctgcacgagcttggcgtctactacctgcccgacgccgagggtgccttccgccgcggcccgggcctgaacttgaccagcggccagtacagggcgcccgtggctggcttctacgctctcgccgccacgctgcacgtggcgctcggggagccgccgaggagggggccgccgcgcccccgggaccacctgcgcctgctcatctgcatccagtcccggtgccagcgcaacgcctccctggaggccatcatgggcctggagagcagcagtgagctcttcaccatctctgtgaatggcgtcctgtacctgcagatggggcagtggacctccgtgttcttggacaacgccagcggctgctccctcacagtgcgcagtggctcccacttcagtgctgtcctcctgggcgtgtgagcggccaccacaggcccttcctctcaggggcaaatggagcacagatctagacaatgtgtggacagtgtcagagtagcagtggccacatggaggaggaggcccacccggaactctgcccacactggccactgcagttcagcccacagagccactgcaggcaggcctacggacgtgacacgcacgctggtggtcccggagccagggttgattcaggacaccatcttgggctcttatccaggaaagaaagagtcggcgtgcctgggggcacctgctagtctccagctgcaggccgactctttcctggcctgctcagcacctgcccagatggcctctgcgtctttcctgtgcccagccccaccttttccacctctcttcatgttctcatggagtgcaaagtgcaccagccagggcccctacctgggagagggtcagctgacgcagggctgagggggctgccacagggacgtacgctgtgtgttcttactgcttagaagggacggggtcactcaccactcccctggtctccatctgggctccttggtcttccctgcccctcccctaaccgtgttctacctgccagtggagctgagcactgcctagcctgggccagaggggcactggacaagggcctttgggggacagtaagtctgggcccagcttctagttctaatgtgtgcaagattaactcacaaattcacctcagaaggcctttccaaatgggagctccactcttcccctcctctgcaaatcttcacagccaaggccccttccaccctctccagaggtggatgagatcccttttcccctcccctctgaggtgctgactcactggtaggagccacccaccagaggaaagatctagaacgtctttacagattggagacagccgggcctgctgacccatcatccgaatagctgaagcagagtcttccaccaggggtgccagggcctggctgggtccagcggctctgggatgagcctcccaagcatctttcccacttgggtggccatgcggcgctgacattggacaggtggtggacgagagatggtcccgagaaagggtggtcttgggagggctggtcccaagcctgctgtgctcctgtggcagtgatggggcctggggatggggacggcagctctcatgaggacacacaggctgtgagcccgcagcctcctcaaatgtagcctcccacattttccccaaagtacaggactgtcccagagtaggtagtgaagaggacaaggccctcggcagcgacctccagggcctcctacctgctgaggaagagttaacccactgcctccccacacaacaggctacgaagaacctggtgcctcaggacctcctgggagccaagctggtctggcaagggcgctcaggcctgggagagaagggagcaatggccagtcaccttcaccttctaactaactagcctccggatgaggtggctgccaccaggcccgaatgatccccaggagcccagcttccaaaccccaacatcgaatcaaacatctccatccccaagtgcagtaacacacaaaaaccaaacactctgccctgggaaaggcctggtgcgattctcagtaggactcacacccaccctacctagaagtactgggctggcctgggtactgcatccgtgtgttttgataagggggtgatgtggccacgcccttatctagatttcactttgtatccactgggcacagatattctagagaacttatctttcactcttgtaaaagccacatatccacatctctttcatttttctcagtgtgttatgcagcaatttattaaagtatttattgtctaataaatactgccaagtggaa

SEQ ID NO: 3 - protein, 354 aa, Homo sapiens
MAPARRPAGARLLLVYAGLLAAAAAGLGSPEPGAPSRSRARREPPPGNELPRGPGESRAGPAARPPEPTAERAHSVDPRDAWMLFVRQSDKGVNGKKRSRGKAKKLKFGLPGPPGPPGPQGPPGPIIPPEALLKEFQLLLKGAVRQRERAEPEPCTCGPAGPVAASLAPVSATAGEDDDDVVGDVLALLAAPLAPGPRAPRVEAAFLCRLRRDALVERRALHELGVYYLPDAEGAFRRGPGLNLTSGQYRAPVAGFYALAATLHVALGEPPRRGPPRPRDHLRLLICIQSRCQRNASLEAIMGLESSSELFTISVNGVLYLQMGQWTSVFLDNASGCSLTVRSGSHFSAVLLGV

SEQ ID NO: 4 - protein, 358 aa, Homo sapiens
MAPARRPAGARLLLVYAGLLAAAAAGLGSPEPGAPSRSRARREPPPGNELPRGPGESRAGPAARPPEPTAERAHSVDPRDAWMLFVRQSDKGVNGKKRSRGKAKKLKVPFQFGLPGPPGPPGPQGPPGPIIPPEALLKEFQLLLKGAVRQRERAEPEPCTCGPAGPVAASLAPVSATAGEDDDDVVGDVLALLAAPLAPGPRAPRVEAAFLCRLRRDALVERRALHELGVYYLPDAEGAFRRGPGLNLTSGQYRAPVAGFYALAATLHVALGEPPRRGPPRPRDHLRLLICIQSRCQRNASLEAIMGLESSSELFTISVNGVLYLQMGQWTSVFLDNASGCSLTVRSGSHFSAVLLGV

SEQ ID NO: 5 - protein, 4 aa, Artificial
  Synthetic 4 amino acids VPFQ
VPFQ

SEQ ID NO: 6 - DNA, 12 bp, Artificial
  Synthetic 12 nucleic acids GTTCCCTTTCAG
gttccctttcag

SEQ ID NO: 7 - protein, 31 aa, Artificial
  Synthetic cryptic peptide
VPFQFGLPGPPGPPGPQGPPGPIIPPEALLK

SEQ ID NO: 8 - DNA, 14 bp, Artificial
  Synthetic ASO
aactgaaagggaac

SEQ ID NO: 9 - DNA, 25 bp, Artificial
  Synthetic ASO
aaagggaaccttggcagtgaggaca

SEQ ID NO: 10 - DNA, 21 bp, Artificial
  Synthetic ASO
accttggcagtgaggacatgt

SEQ ID NO: 11 - protein, 18 aa, Artificial
  Synthetic peptide obtained by MS
ALHELGVYYLPDAEGAFR

(19) United States
(12) Patent Application Publication
(10) Pub. No.: US 2022/0119516 A1
(43) Pub. Date: Apr. 21, 2022

(54) VARIANTS OF ERYTHROFERRONE AND THEIR USE

(71) Applicants: INSERM (Institut National de la Santé et de la Recherche Médicale), Paris (FR); Centre Natioanl de la Recherche Scientifique (CNRS), Paris (FR); Université de Paris, Paris (FR); Assistance Publique-Hôpitaux de Paris (APHP), Paris (FR); UNIVERSITÉ PAUL SABATIER TOULOUSE III, Toulouse (FR); ECOLE NATIONALE VÉTÉRINAIRE DE TOULOUSE, Toulouse (FR); Institut Curie, Villejuif Cedex (FR)

(72) Inventors: Michaela FONTENAY, Paris (FR); Olivier KOSMIDER, Paris (FR); François GUILLONNEAU, Paris (FR); Léon KAUTZ, Toulouse cedex 03 (FR); Samar ALSAFADI, Paris (FR); Carine LEFEVRE, Paris (FR); Marc-Henri STERN, Paris (FR); Alexandre HOUY, Paris (FR)

(21) Appl. No.: 17/423,775

(22) PCT Filed: Jan. 15, 2020
(86) PCT No.: PCT/EP2020/050946

§ 371 Date: Jul. 16, 2021

(30) Foreign Application Priority Data
Jan. 16, 2019 (EP) 19305047.3

(51) Int. Cl. C07K 16/26; C07K 14/575; C12N 15/113; A61P 7/06; C12Q 1/6883

(52) U.S. Cl. CPC C07K 16/26; C07K 14/575; C12N 15/1136; A61K 39/00; C12Q 1/6883; C12N 2310/11; A61P 7/06

(57) ABSTRACT

The invention relates to the domain of anemia, iron overload and myeloid malignancy. The inventors identify a variant transcript of ERFE specific of SF3B1^{MUT} MDS that contributes to increased concentration of ERFE protein leading to hepcidin suppression and iron accumulation in patients. This transcript contains an in-frame added intronic sequence of 12 nucleotides not inducing a stop codon that may be translated into a variant protein with an additional 4 amino acids. By using deep mass spectrometry, they identified a peptide corresponding to the added polypeptide VPQF (SEQ ID NO: 5) demonstrating the active production of a variant protein by bone marrow erythroblasts of patients with a SF3B1-mutated MDS. This variant can be used as a pertinent biomarker of clonal erythropoiesis for monitoring treatments of anemia in SF3B1^{MUT} patients. Thus, the invention relates to a variant of the transcript of ERFE and its use in diagnosing and monitoring of anemia and iron overload in patient with a myeloid malignancy with at least one mutation in the SF3B1 gene.

### FIELD OF THE INVENTION

The present invention relates to a variant of the transcript of ERFE and its use in diagnosing and monitoring of anemia and iron overload in patient with a myeloid malignancy with at least one mutation in the SF3B1 gene.

### BACKGROUND OF THE INVENTION

Myelodysplastic syndromes are clonal hematopoietic stem cell disorders affecting patients in the elderly with a propensity in 40% of cases to evolution into acute myeloid leukemia (defined by ≥20% of bone marrow blast cells) or in the rest of the cases to bone marrow failure. Patients suffer from peripheral blood cytopenias mainly anemia (in 80% of cases), and less frequently neutropenia and/or thrombocytopenia mostly associated with comorbidities linked to age and possibly to the onset of clonal hematopoiesis. MDS are heterogeneous and the World Health Organization recognizes seven subtypes in his 2016 classification (1). The inter-individual heterogeneity increases since the discovery of distinct combinations of recurrent mutations in the bone marrow hematopoietic cells. These mutations affect genes involved in the epigenetic regulation of transcription (TET2, IDH1/2, DNMT3A, ASXL1, EZH2), splicing factors (SF3B1, SRSF2, U2AF1, ZRSR2), cohesins (STAG2), transcription factor (RUNX1, TP53) and signaling molecules (KIT, CBL, NRAS, KRAS). Each of these mutations are potentially initiating events detectable in the more immature stem and progenitor cells of the bone marrow and are transmitted to their erythroid and granulo-monocytic progeny and sometimes to B lymphocytes (2). Risk factors are the number and intensity of cytopenias, the percentage of bone marrow blasts and the type and number of cytogenetic abnormalities all referred into the revised International prognosis scoring system (R-IPSS; ref. 3). Patients are recognized as very low, low, intermediate, high and very high risk. A new prognosis scoring system including the mutational status is under study. The aim of treatments in lower risk patients is to cure cytopenias while it is to stop the progression of leukemia in higher risk patients. The first line treatments are erythropoiesis-stimulating agents (ESA) and transfusions in case of failure. ESA comprise recombinant erythropoietin (Epo), including epoetin alpha, epoetin beta and darbepoetin and biosimilars like Retacrit (epoetin zeta) (for review ref 4).

However, 50% of anemic patients exhibit primary or secondary resistance to ESA highlighting the requirement for other therapeutic options. Lenalidomide has been successfully used in patients suffering from a particular MDS subtype with a 5q deletion (5). Lenalidomide was also transiently efficient in association with ESA in non del5q MDS patients (6, 7). By contrast, hypomethylating agents, which efficiently prolong survival in higher risk patients are poorly efficient in the treatment of anemia (GFM aza/Epo). Recently, TGF-β family member ligand trap, luspatercept has been reported as an effective agent for the treatment of anemia in lower risk patients. This treatment has been first tested in mice models of β-thalassemia or MDS two models of ineffective erythropoiesis. The results demonstrate a rescue of late stages of erythropoiesis and an improvement of iron parameters (8, 9). A recent report of the phase II study on lower risk MDS highlights a response rate over 50% and more than 70% in patients with myelodysplastic syndromes with ring sideroblasts (MDS-RS).

Myelodysplastic syndromes with ring sideroblasts (MDS-RS) are clonal hematopoietic stem cell (HSC) disorders with a prominent erythroid dysplasia of the bone marrow (BM) responsible for a macrocytic anemia. Mitochondrial iron accumulation and apoptosis of mature erythroblasts cause ineffective erythropoiesis (10, 11). In contrast to other MDS subtypes, patients with MDS-RS exhibit signs of systemic iron accumulation that is reflected by increased ferritin and non-transferrin bound iron levels before they become transfusion-dependent and develop parenchymal iron overload thereafter (12, 13). Systemic iron overload is a source of LPI (labile plasma iron) which may lead to the generation of reactive oxygen species in the cells. This contributes to a decreased survival and DNA damage in hematopoietic progenitor therefore, aggravating ineffective erythropoiesis in the bone marrow. Iron chelation inhibits the consequences of iron overload and improves erythropoiesis and life expectancy in MDS patients (14, 15).

Splicing factor gene SF3B1 is mutated in ˜90% of MDS-RS and the diagnosis is considered whenever the gene is mutated, even if the percentage of RS is relatively low between 5 and 15% (16-18). Mutations arise in the HSC (2, 19-20). Aberrant splicing events are reported in MDS and other SF3B1-driven cancers including uveal melanoma and chronic lymphocytic leukemia (CLL) (21-24). The selection of an alternative branch site (BS) resulting in the use of a cryptic 3′ splice site (ss) is the most frequently detected abnormality. Computational analysis revealed that the majority of cryptic 3′ss are located upstream of canonical 3′ss at nucleotide distances that are not multiple of 3 suggesting that the aberrant transcripts would likely contain a premature termination codon (PTC) and be degraded by the non-sense mediated decay (NMD). It has been predicted that half of the aberrantly spliced transcripts in SF3B1-mutated cells are NMD-sensitive and the canonical isoforms are down-regulated. For instance, ABCB7 transcript encoding a mitochondrial transporter involved in the export of Fe—S clusters is aberrantly spliced and undergoes NMD (21, 25-27). It is also possible that NMD-insensitive aberrant transcripts are translated into proteins with altered function (21). How these aberrant splicing events contribute to the disease phenotype and in particular to systemic iron overload is unclear.

In contrast with other MDS subtypes, MDS-RS are associated with lower levels of the iron homeostasis regulator, hepcidin, and as a consequence, the absorption of iron by duodenal enterocytes and its release from erythrophagocytic macrophages may be increased (28-33). Inappropriately low hepcidin levels in MDS-RS could depend on the degree of ineffective erythropoiesis linked to impaired iron incorporation into heme because of mitochondrial iron trapping, or to increased expression of hepcidin repressor (34). Growth differentiation factor 15 (GDF-15) and twisted gastrulation (TWSG1), two members of the transforming growth factor-β superfamily have been proposed as pathological suppressors of hepcidin in ineffective erythropoiesis (35, 36). More recently, erythroferrone (ERFE), a C1q-tumor necrosis factor-related family of proteins (CTRP) member has been described as a major erythroid regulator of hepcidin and involved in the pathological suppression of hepcidin in patients with β-thalassemia (37, 38).

Treatments of anemia failed in 50-70% of cases. Erythroid response is evaluated using IWG criteria (2006) including the number of RBC units per 8 weeks and Hb level, meaning that erythropoiesis is evaluated globally. Transfusion independency is defined by the absence of RBC transfusion for 8 wk or longer following treatment and hematological improvement is defined as a relevant reduction of units of RBC transfusions by an absolute number of at least 4 RBC transfusions/8 wk compared with the pretreatment transfusion number in the previous 8 wk. Only RBC transfusions given for a Hb of ≤9.0 g/dL pretreatment will count in the RBC transfusion response evaluation or an increase of Hb level ≥1.5 g/dL (39). The Nordic group has proposed a predictive score of the response to ESA based on serum Epo level, and the number of transfusions before treatment (40). This score was validated and improved by others including the French Group of Myelodysplasia (Park S, Blood 2006). But in the real life, and despite the wide diffusion of the Nordic score, the response rate to ESA remained around 50% (41). Therefore, an appropriate score of prediction is still lacking for alternative therapeutic options. To better understand the mechanism of response, an accurate evaluation of pathological bone marrow erythropoiesis at diagnosis and during evolution and treatments. The identification of a hallmark of pathological erythropoiesis may help the design of a biomarker of response to the treatments.

### SUMMARY OF THE INVENTION

In the present study, the inventors identify a variant transcript of ERFE specific of SF3B1^{MUT} MDS that contributes to increased concentration of ERFE protein leading to hepcidin suppression and iron accumulation in patients. This transcript contains an in-frame added intronic sequence of 12 nucleotides not inducing a stop codon that may be translated into a variant protein with an additional 4 amino acids. By using deep mass spectrometry, they identified a peptide corresponding to the added polypeptide VPQF (SEQ ID NO: 5) demonstrating the active production of a variant protein by bone marrow erythroblasts of patients with a SF3B1-mutated MDS. The recombinant variant protein ERFE^{VPFQ} represses the expression of hepcidin mRNA in the same extent than the wild-type recombinant ERFE. This variant is a pertinent biomarker of clonal erythropoiesis for monitoring treatments of anemia in SF3B1^{MUT} patients.

Thus, the present invention relates to a variant of the transcript of ERFE and its use in diagnosing and monitoring of anemia and iron overload in patient with a myeloid malignancy with at least one mutation in the SF3B1 gene. Particularly, the invention is defined by its claims.

### DETAILED DESCRIPTION OF THE INVENTION

### Variants of the Invention

A first aspect of the invention relates to a variant of the transcript of ERFE having at least 70% of homology with the nucleic acid sequence SEQ ID NO: 2 and to a variant of the protein ERFE having at least 70% of homology with the amino acid sequence SEQ ID NO: 4.

According to the invention, the variant of the invention can have 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of homology with the SEQ ID NO: 2 or the SEQ ID NO: 4.

In one embodiment the variant of the transcript of ERFE has a nucleic acid sequence SEQ ID NO: 2 (ERFE⁺¹²).

In another embodiment, the variant of the protein ERFE has an amino acid sequence SEQ ID NO: 4 (ERFE^{VPFQ}).

In another embodiment, the variant of the protein of ERFE has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of homology with the SEQ ID NO:3 and comprises in the sequence the 4 amino acids VPFQ of SEQ ID NO: 5.

In another embodiment, the variant of the protein of ERFE has at least 70% of homology with the SEQ ID NO:3 and comprises the 4 amino acids VPFQ of SEQ ID NO: 5.

In another embodiment, the variant of the protein of ERFE has at least 70% of homology with the SEQ ID NO:3 and comprises in the sequence SEQ ID NO:3 the 4 amino acids VPFQ of SEQ ID NO: 5.

In another embodiment, the variant of the protein of ERFE has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of homology with the SEQ ID NO:3 and comprises in the sequence the 4 amino acids VPFQ of SEQ ID NO: 5.

In another embodiment, the variant of the transcript of ERFE has at least 70% of homology with the SEQ ID NO:1 and comprises in the sequence the 12 nucleic acids GTTCCCTTTCAG of SEQ ID NO: 6.

In another embodiment, the variant of the protein ERFE comprises at least the amino acids sequence of SEQ ID NO: 7 in its amino acids sequence.

In another embodiment, the variant of the protein ERFE has an amino acid sequence SEQ ID NO: 7.

According to the invention, the term “variants” (in plural) denote all the variants of the transcript ERFE and all the variant of the protein ERFE.

As used herein the term “homology” used according to the invention has the same meaning that the term “identity”.

### Methods of Diagnosing and Monitoring of the Invention

A second aspect of the invention relates to a method for diagnosing an anemia in a patient suffering from a myeloid malignancy with at least one mutation in the SF3B1 gene comprising determining, in a sample obtained from the patient, the expression of a variant of the ERFE transcript or a variant of the ERFE protein wherein the detection of such variants indicate that the patient suffer from an anemia with at least one mutation in the SF3B1 gene.

The inventors showed that when specific treatments of anemia in patient suffering from a myeloid malignancy with at least one mutation in the SF3B1 gene is used, the quantity of the variant (transcript or protein) can be significantly modified. This modification in the expression of the variant means that the drug directly or indirectly targets the SF3B1-mutated progenitors and/or erythroid precursors in the bone marrow. Thus, determining the expression of the variant of ERFE allows to explore the mechanism of action of a drug. For example, when some drugs like the lenalidomide are used to treat an anemia, the variant is significantly decreased, the SF3B1-mutated progenitors and/or erythroid precursors are targeted and the treatment (the lenalidomide) is effective (see Results part). In contrary, when a TGF-β family member ligand trap like the luspatercept is used to treat an anemia, the variant is not significantly decreased and the treatment is effective but the SF3B1-mutated progenitors and/or erythroid precursors are not targeted.

In other words, when a drug which target the terminal erythropoiesis like the luspatercept is used to treat an anemia, the variant is not significantly decreased and the treatment is effective but the SF3B1-mutated progenitors and/or erythroid precursors are not targeted.

According to the invention, the terms “terminal erythropoiesis” denote all the cells of the terminal erythropoiesis like the proerythroblast, the basophilic erythroblast, the polychromatophilic erythroblast and the orthochromatic erythroblast.

Thus, a third aspect of the invention relates to a method which allows to indicate if a treatment of a anemia will or not target the SF3B1-mutated progenitors and/or erythroid precursors in a patient suffering from a myeloid malignancy with at least one mutation in the SF3B1 gene comprising determining, in a sample obtained from the patient, the expression of a variant of the ERFE transcript or a variant of the ERFE protein wherein the detection of such variants indicates that said treatment is effective or not in targeting the clonal/abnormal SF3B1-mutated erythropoiesis.

In one embodiment, the treatment of anemia can be the lenalidomide.

Thus, in a particular embodiment, the invention relates to a method which allows to indicate if the treatment by the lenalinomide will or not target the SF3B1-mutated progenitors and/or erythroid precursors in a patient suffering from a myeloid malignancy with at least one mutation in the SF3B1 gene comprising determining, in a sample obtained from the patient, the expression of a variant of the ERFE transcript or a variant of the ERFE protein wherein the detection of such variants indicates that said treatment is effective to targeting the clonal/abnormal SF3B1-mutated erythropoiesis.

As used herein, the terms “targeting the clonal/abnormal SF3B1-mutated erythropoiesis” denotes the fact of eliminate/deplete the clonal/abnormal SF3B1-mutated erythropoiesis. Thus, the terms “said treatment is effective to targeting the clonal/abnormal SF3B1-mutated erythropoiesis” denotes that the treatment allows the elimination/depletion of the clonal/abnormal SF3B1-mutated erythropoiesis. Thus, the terms “the SF3B1-mutated progenitors and/or erythroid precursors are not targeted” dente that the SF3B1-mutated progenitors and/or erythroid precursors are not eliminated/depleted.

As used herein, the terms “progenitors and/or erythroid precursors” denote all the cells of the erythropoiesis (the process which gives the erythrocytes) starting from the hematopoietic stem cells to the reticulocytes. This terms denotes for example the common myeloid progenitor, the megakaryocytic-erythroid progenitor, the burst-forming unit-erythroid, the colony-forming unit-erythroid, the proerythroblast, the basophilic erythroblast, the polychromatophilic erythroblast and the orthochromatic erythroblast. According to the invention, the terms “clonal erythropoiesis” or “clonal/abnormal SF3B1-mutated erythropoiesis” has the same meaning that “progenitors and/or erythroid precursors”.

As used herein, the terms “SF3B1-mutated progenitors and/or erythroid precursors” denote all the cells of the erythropoiesis having at least one mutation in the SF3B1 gene.

Another aspect of the invention relates to a method of monitoring a treatment of anemia by lenalinomide in a patient suffering from a myeloid malignancy with at least one mutation in SF3B1 gene comprising determining, in a sample obtained from the patient, i) the expression level of a variant of the ERFE transcript or of a variant of the ERFE protein before and after the treatment by lenalinomide, ii) comparing said the expression levels obtained before and after the treatment by lenalinomide wherein when the expression level of the variants obtained after the treatment by lenalinomide is reduced compared to a the expression level of the variants obtained before the treatment, this indicates that the SF3B1-mutated erythropoiesis is decreased and that the patient responds to the treatment by lenalinomide.

According to the invention, when the variant is transcript variant of ERFE, the sample is particularly the bone marrow mononuclear cells and when the variant is the protein variant of ERFE, the sample is particularly the peripheral blood serum.

The inventors also showed that the variants of ERFE could be used as a biomarker in systemic iron overload.

Thus, a fourth aspect of the invention relates to a method for diagnosing a systemic iron overload in a patient suffering from a myeloid malignancy with at least one mutation in the SF3B1 gene comprising determining, in a sample obtained from the patient, the expression of a variant of the ERFE transcript or a variant of the ERFE protein wherein the detection of such variants indicates that said patient has a systemic iron overload.

In another aspect, the invention relates to a method for predicting a parenchymal iron overload in liver and heart in a patient suffering from a myeloid malignancy with at least one mutation in the SF3B1 gene comprising determining, in a sample obtained from the patient, the expression of a variant of the ERFE transcript or a variant of the ERFE protein wherein the detection of such variants indicates that said patient will have a predisposition to parenchymal iron overload in liver and heart.

According to the invention, the terms ‘systemic iron overload” also denotes hyperferritinemia.

Another aspect of the invention relates to a method of monitoring a treatment of systemic iron overload in a patient suffering from a myeloid malignancy with at least one mutation in the SF3B1 gene comprising determining, in a sample obtained from the patient, i) the expression level of a variant of the ERFE transcript or of a variant of the ERFE protein before and after the treatment, ii) comparing said the expression levels obtained before and after the treatment wherein when the expression level of the variants obtained after the treatment is reduced compared to a the expression level of the variants obtained before the treatment, this indicates that the patient responds to the treatment.

In other terms, when the expression level of the variants decreases, the treatment of iron overload is effective and when the expression level of the variants does not decrease or increases, the treatment is not effective and the systemic iron overload aggravates.

In another aspect, the ratio between the quantity (concentration) of a variant of ERFE (noted as ERFE^{V}) (transcript or protein) and a variant of ERFE (noted as ERFE^{V}) (transcript or protein) plus ERFE wild type (transcript or protein, noted as ERFE^{WT}) can be used for the methods of the invention (ERFEVERFE^{V}+ERFE^{WT}). Particularly, the ratio is done with the transcript variant of ERFE and is used to monitor a treatment of anemia.

A fifth aspect of the invention relates to a method of monitoring a treatment of anemia in a patient suffering from a myeloid malignancy with at least one mutation in the SF3B1 gene comprising determining, in a sample obtained from the patient, i) the ratio ERFE^{V}/ERFE^{V}+ERFE^{WT} before and after the treatment, ii) comparing said ratios obtained before and after the treatment wherein when the ratio is reduced compared to a predetermined reference value, the patient will respond to the treatment and when it remained unchanged compared to a predetermined reference value, the patient will not respond to the treatment.

In a particular embodiment, the sample is bone marrow mononuclear cells of the patient.

The invention also relates to a variant of ERFE (variant of the transcript or of the protein) as a biomarker of clonal erythropoiesis.

According all aspects of the invention, the terms “myeloid malignancy with at least one mutation in the I gene” denotes a myelodysplastic syndrome (MDS) with at least one mutation in the SF3B1 gene, a myelodysplastic syndrome with ring sideroblasts, a myelodysplastic syndrome with ring sideroblasts in 90% of cases, a myelodysplastic syndromes with ring sideroblasts (MDS-RS) with at least one mutation in the SF3B1 gene, an acute myeloid leukemia (de novo or secondary) with at least one mutation in the SF3B1 gene, a myeloproliferative neoplasms with at least one mutation in the SF3B1 gene and a mixed myeloproliferative/myelodysplastic syndrome with at least one mutation in the SF3B1 gene (including anemia with ring sideroblasts and thrombocytosis, and others).

As used herein and according to all aspects of the invention, the term “sample” denotes, bone marrow mononuclear cells, bone marrow serum, peripheral blood, mononuclear cells, peripheral-blood serum and peripheral blood plasma.

As used herein, the term “patient” refers to an individual who is being managed for anaemia disease or who is susceptible to develop an anaemia or with an iron overload or a patient with a myeloid malignancy and particularly a myeloid malignancy with at least one mutation in the SF3B1 gene.

According to all aspects of the invention, the variant of ERFE can be a variant of the transcript of ERFE like ERFE⁺¹² (SEQ ID NO:2) or a variant of the protein of ERFE like ERFE^{VPFQ} (SEQ ID NO:4).

According to all aspects of the invention, the quantity (concentration) of all the variants described in the invention can be used in the context of the methods of the invention.

As used herein, the term ‘ERFE^{VPFQ}” denotes any variant of ERFE. Said variant can be a variant of the transcript or of the protein of ERFE.

As used herein, the term ‘ERFE^{WT}” denotes the wild version of ERFE.

As used herein, the term ‘ERFE⁺¹²” denotes the transcript variant of ERFE containing 12 nucleic acids in more compare to the wild type nucleotidic sequence of ERFE (SEQ ID NO: 2).

As used herein, the term “ERFE^{VPFQ}” denotes the protein variant of ERFE containing 4 amino acids in more compare to the wild type amino acids sequence of ERFE (SEQ ID NO:4).

As used herein the term “ERFE” also known as “Erythroferrone” refers to a protein produced by erythroblasts which inhibits the action of hepcidin, and so increases the amount of iron available for hemoglobin synthesis. The sequence of said gene can be found under the Ensembl accession number ENSG00000178752.

Nucleic acids sequence of the ERFE (SEQ ID NO: 1):
GACGCCGCGCTCGGAGCCGCGAGGGAACCGCCGCCCGCATGGCCCCGGCC
CGCCGCCCCGCCGGAGCCCGCCTGCTGCTCGTCTACGCGGGCCTGCTGGCCGCCG
CCGCCGCGGGCCTGGGGTCCCCGGAGCCTGGGGCGCCCTCGAGGAGCCGCGCCC
GCAGGGAGCCGCCGCCCGGGAACGAGCTGCCCCGGGGCCCCGGGGAGAGCCGCG
CGGGGCCGGCCGCTCGTCCGCCGGAGCCCACCGCTGAGCGTGCACACAGCGTCG
ACCCCCGGGACGCCTGGATGCTCTTCGTCAGGCAGAGTGACAAGGGTGTCAATGG
CAAGAAGAGGAGCAGGGGCAAGGCCAAGAAGCTGAAGTTCGGCTTGCCAGGGCC
CCCTGGGCCTCCCGGTCCCCAGGGCCCCCCAGGCCCCATCATCCCACCCGAGGCG
CTGCTGAAGGAGTTCCAGCTGCTGCTGAAAGGTGCGGTGCGGCAGCGGGAGCGC
GCGGAGCCCGAACCCTGTACGTGTGGCCCCGCCGGGCCGGTCGCTGCGAGCCTCG
CCCCGGTCTCGGCCACCGCCGGGGAGGACGACGACGACGTGGTGGGGGACGTGC
TGGCACTGCTGGCCGCGCCCCTGGCCCCGGGGCCGCGGGCGCCGCGCGTGGAGG
CCGCTTTCCTCTGCCGCCTGCGCCGGGACGCGTTGGTGGAGCGGCGCGCGCTGCA
CGAGCTTGGCGTCTACTACCTGCCCGACGCCGAGGGTGCCTTCCGCCGCGGCCCG
GGCCTGAACTTGACCAGCGGCCAGTACAGGGCGCCCGTGGCTGGCTTCTACGCTC
TCGCCGCCACGCTGCACGTGGCGCTCGGGGAGCCGCCGAGGAGGGGGCCGCCGC
GCCCCCGGGACCACCTGCGCCTGCTCATCTGCATCCAGTCCCGGTGCCAGCGCAA
CGCCTCCCTGGAGGCCATCATGGGCCTGGAGAGCAGCAGTGAGCTCTTCACCATC
TCTGTGAATGGCGTCCTGTACCTGCAGATGGGGCAGTGGACCTCCGTGTTCTTGG
ACAACGCCAGCGGCTGCTCCCTCACAGTGCGCAGTGGCTCCCACTTCAGTGCTGT
CCTCCTGGGCGTGTGAGCGGCCACCACAGGCCCTTCCTCTCAGGGGCAAATGGAG
CACAGATCTAGACAATGTGTGGACAGTGTCAGAGTAGCAGTGGCCACATGGAGG
AGGAGGCCCACCCGGAACTCTGCCCACACTGGCCACTGCAGTTCAGCCCACAGA
GCCACTGCAGGCAGGCCTACGGACGTGACACGCACGCTGGTGGTCCCGGAGCCA
GGGTTGATTCAGGACACCATCTTGGGCTCTTATCCAGGAAAGAAAGAGTCGGCGT
GCCTGGGGGCACCTGCTAGTCTCCAGCTGCAGGCCGACTCTTTCCTGGCCTGCTC
AGCACCTGCCCAGATGGCCTCTGCGTCTTTCCTGTGCCCAGCCCCACCTTTTCCAC
CTCTCTTCATGTTCTCATGGAGTGCAAAGTGCACCAGCCAGGGCCCCTACCTGGG
AGAGGGTCAGCTGACGCAGGGCTGAGGGGGCTGCCACAGGGACGTACGCTGTGT
GTTCTTACTGCTTAGAAGGGACGGGGTCACTCACCACTCCCCTGGTCTCCATCTGG
GCTCCTTGGTCTTCCCTGCCCCTCCCCTAACCGTGTTCTACCTGCCAGTGGAGCTG
AGCACTGCCTAGCCTGGGCCAGAGGGGCACTGGACAAGGGCCTTTGGGGGACAG
TAAGTCTGGGCCCAGCTTCTAGTTCTAATGTGTGCAAGATTAACTCACAAATTCA
CCTCAGAAGGCCTTTCCAAATGGGAGCTCCACTCTTCCCCTCCTCTGCAAATCTTC
ACAGCCAAGGCCCCTTCCACCCTCTCCAGAGGTGGATGAGATCCCTTTTCCCCTCC
CCTCTGAGGTGCTGACTCACTGGTAGGAGCCACCCACCAGAGGAAAGATCTAGA
ACGTCTTTACAGATTGGAGACAGCCGGGCCTGCTGACCCATCATCCGAATAGCTG
AAGCAGAGTCTTCCACCAGGGGTGCCAGGGCCTGGCTGGGTCCAGCGGCTCTGG
GATGAGCCTCCCAAGCATCTTTCCCACTTGGGTGGCCATGCGGCGCTGACATTGG
ACAGGTGGTGGACGAGAGATGGTCCCGAGAAAGGGTGGTCTTGGGAGGGCTGGT
CCCAAGCCTGCTGTGCTCCTGTGGCAGTGATGGGGCCTGGGGATGGGGACGGCA
GCTCTCATGAGGACACACAGGCTGTGAGCCCGCAGCCTCCTCAAATGTAGCCTCC
CACATTTTCCCCAAAGTACAGGACTGTCCCAGAGTAGGTAGTGAAGAGGACAAG
GCCCTCGGCAGCGACCTCCAGGGCCTCCTACCTGCTGAGGAAGAGTTAACCCACT
GCCTCCCCACACAACAGGCTACGAAGAACCTGGTGCCTCAGGACCTCCTGGGAGC
CAAGCTGGTCTGGCAAGGGCGCTCAGGCCTGGGAGAGAAGGGAGCAATGGCCAG
TCACCTTCACCTTCTAACTAACTAGCCTCCGGATGAGGTGGCTGCCACCAGGCCC
GAATGATCCCCAGGAGCCCAGCTTCCAAACCCCAACATCGAATCAAACATCTCCA
TCCCCAAGTGCAGTAACACACAAAAACCAAACACTCTGCCCTGGGAAAGGCCTG
GTGCGATTCTCAGTAGGACTCACACCCACCCTACCTAGAAGTACTGGGCTGGCCT
GGGTACTGCATCCGTGTGTTTTGATAAGGGGGTGATGTGGCCACGCCCTTATCTA
GATTTCACTTTGTATCCACTGGGCACAGATATTCTAGAGAACTTATCTTTCACTCT
TGTAAAAGCCACATATCCACATCTCTTTCATTTTTCTCAGTGTGTTATGCAGCAAT
TTATTAAAGTATTTATTGTCTAATAAATACTGCCAAGTGGAA
Nucleic acids sequence of the ERFE⁺¹² (SEQ ID NO: 2):
GACGCCGCGCTCGGAGCCGCGAGGGAACCGCCGCCCGCATGGCCCCGGCC
CGCCGCCCCGCCGGAGCCCGCCTGCTGCTCGTCTACGCGGGCCTGCTGGCCGCCG
CCGCCGCGGGCCTGGGGTCCCCGGAGCCTGGGGCGCCCTCGAGGAGCCGCGCCC
GCAGGGAGCCGCCGCCCGGGAACGAGCTGCCCCGGGGCCCCGGGGAGAGCCGCG
CGGGGCCGGCCGCTCGTCCGCCGGAGCCCACCGCTGAGCGTGCACACAGCGTCG
ACCCCCGGGACGCCTGGATGCTCTTCGTCAGGCAGAGTGACAAGGGTGTCAATGG
CAAGAAGAGGAGCAGGGGCAAGGCCAAGAAGCTGAAGGTTCCCTTTCAGTTCGG
CTTGCCAGGGCCCCCTGGGCCTCCCGGTCCCCAGGGCCCCCCAGGCCCCATCATC
CCACCCGAGGCGCTGCTGAAGGAGTTCCAGCTGCTGCTGAAAGGTGCGGTGCGG
CAGCGGGAGCGCGCGGAGCCCGAACCCTGTACGTGTGGCCCCGCCGGGCCGGTC
GCTGCGAGCCTCGCCCCGGTCTCGGCCACCGCCGGGGAGGACGACGACGACGTG
GTGGGGGACGTGCTGGCACTGCTGGCCGCGCCCCTGGCCCCGGGGCCGCGGGCG
CCGCGCGTGGAGGCCGCTTTCCTCTGCCGCCTGCGCCGGGACGCGTTGGTGGAGC
GGCGCGCGCTGCACGAGCTTGGCGTCTACTACCTGCCCGACGCCGAGGGTGCCTT
CCGCCGCGGCCCGGGCCTGAACTTGACCAGCGGCCAGTACAGGGCGCCCGTGGC
TGGCTTCTACGCTCTCGCCGCCACGCTGCACGTGGCGCTCGGGGAGCCGCCGAGG
AGGGGGCCGCCGCGCCCCCGGGACCACCTGCGCCTGCTCATCTGCATCCAGTCCC
GGTGCCAGCGCAACGCCTCCCTGGAGGCCATCATGGGCCTGGAGAGCAGCAGTG
AGCTCTTCACCATCTCTGTGAATGGCGTCCTGTACCTGCAGATGGGGCAGTGGAC
CTCCGTGTTCTTGGACAACGCCAGCGGCTGCTCCCTCACAGTGCGCAGTGGCTCC
CACTTCAGTGCTGTCCTCCTGGGCGTGTGAGCGGCCACCACAGGCCCTTCCTCTCA
GGGGCAAATGGAGCACAGATCTAGACAATGTGTGGACAGTGTCAGAGTAGCAGT
GGCCACATGGAGGAGGAGGCCCACCCGGAACTCTGCCCACACTGGCCACTGCAG
TTCAGCCCACAGAGCCACTGCAGGCAGGCCTACGGACGTGACACGCACGCTGGT
GGTCCCGGAGCCAGGGTTGATTCAGGACACCATCTTGGGCTCTTATCCAGGAAAG
AAAGAGTCGGCGTGCCTGGGGGCACCTGCTAGTCTCCAGCTGCAGGCCGACTCTT
TCCTGGCCTGCTCAGCACCTGCCCAGATGGCCTCTGCGTCTTTCCTGTGCCCAGCC
CCACCTTTTCCACCTCTCTTCATGTTCTCATGGAGTGCAAAGTGCACCAGCCAGGG
CCCCTACCTGGGAGAGGGTCAGCTGACGCAGGGCTGAGGGGGCTGCCACAGGGA
CGTACGCTGTGTGTTCTTACTGCTTAGAAGGGACGGGGTCACTCACCACTCCCCT
GGTCTCCATCTGGGCTCCTTGGTCTTCCCTGCCCCTCCCCTAACCGTGTTCTACCT
GCCAGTGGAGCTGAGCACTGCCTAGCCTGGGCCAGAGGGGCACTGGACAAGGGC
CTTTGGGGGACAGTAAGTCTGGGCCCAGCTTCTAGTTCTAATGTGTGCAAGATTA
ACTCACAAATTCACCTCAGAAGGCCTTTCCAAATGGGAGCTCCACTCTTCCCCTCC
TCTGCAAATCTTCACAGCCAAGGCCCCTTCCACCCTCTCCAGAGGTGGATGAGAT
CCCTTTTCCCCTCCCCTCTGAGGTGCTGACTCACTGGTAGGAGCCACCCACCAGA
GGAAAGATCTAGAACGTCTTTACAGATTGGAGACAGCCGGGCCTGCTGACCCATC
ATCCGAATAGCTGAAGCAGAGTCTTCCACCAGGGGTGCCAGGGCCTGGCTGGGTC
CAGCGGCTCTGGGATGAGCCTCCCAAGCATCTTTCCCACTTGGGTGGCCATGCGG
CGCTGACATTGGACAGGTGGTGGACGAGAGATGGTCCCGAGAAAGGGTGGTCTT
GGGAGGGCTGGTCCCAAGCCTGCTGTGCTCCTGTGGCAGTGATGGGGCCTGGGGA
TGGGGACGGCAGCTCTCATGAGGACACACAGGCTGTGAGCCCGCAGCCTCCTCA
AATGTAGCCTCCCACATTTTCCCCAAAGTACAGGACTGTCCCAGAGTAGGTAGTG
AAGAGGACAAGGCCCTCGGCAGCGACCTCCAGGGCCTCCTACCTGCTGAGGAAG
AGTTAACCCACTGCCTCCCCACACAACAGGCTACGAAGAACCTGGTGCCTCAGGA
CCTCCTGGGAGCCAAGCTGGTCTGGCAAGGGCGCTCAGGCCTGGGAGAGAAGGG
AGCAATGGCCAGTCACCTTCACCTTCTAACTAACTAGCCTCCGGATGAGGTGGCT
GCCACCAGGCCCGAATGATCCCCAGGAGCCCAGCTTCCAAACCCCAACATCGAAT
CAAACATCTCCATCCCCAAGTGCAGTAACACACAAAAACCAAACACTCTGCCCTG
GGAAAGGCCTGGTGCGATTCTCAGTAGGACTCACACCCACCCTACCTAGAAGTAC
TGGGCTGGCCTGGGTACTGCATCCGTGTGTTTTGATAAGGGGGTGATGTGGCCAC
GCCCTTATCTAGATTTCACTTTGTATCCACTGGGCACAGATATTCTAGAGAACTTA
TCTTTCACTCTTGTAAAAGCCACATATCCACATCTCTTTCATTTTTCTCAGTGTGTT
ATGCAGCAATTTATTAAAGTATTTATTGTCTAATAAATACTGCCAAGTGGAA
Amino acids sequence of the ERFE (SEQ ID NO: 3):
MAPARRPAGARLLLVYAGLLAAAAAGLGSPEPGAPSRSRARREPPPGNELPR
GPGESRAGPAARPPEPTAERAHSVDPRDAWMLFVRQSDKGVNGKKRSRGKAKKLKF
GLPGPPGPPGPQGPPGPIIPPEALLKEFQLLLKGAVRQRERAEPEPCTCGPAGPVAASL
APVSATAGEDDDDVVGDVLALLAAPLAPGPRAPRVEAAFLCRLRRDALVERRALHE
LGVYYLPDAEGAFRRGPGLNLTSGQYRAPVAGFYALAATLHVALGEPPRRGPPRPRD
HLRLLICIQSRCQRNASLEAIMGLESSSELFTISVNGVLYLQMGQWTSVFLDNASGCSL
TVRSGSHFSAVLLGV
Amino acids sequence of the ERFE^{VPFQ} (SEQ ID NO: 4):
MAPARRPAGARLLLVYAGLLAAAAAGLGSPEPGAPSRSRARREPPPGNELPR
GPGESRAGPAARPPEPTAERAHSVDPRDAWMLFVRQSDKGVNGKKRSRGKAKKLK
VPFQFGLPGPPGPPGPQGPPGPIIPPEALLKEFQLLLKGAVRQRERAEPEPCTCGPAGPV
AASLAPVSATAGEDDDDVVGDVLALLAAPLAPGPRAPRVEAAFLCRLRRDALVERR
ALHELGVYYLPDAEGAFRRGPGLNLTSGQYRAPVAGFYALAATLHVALGEPPRRGPP
RPRDHLRLLICIQSRCQRNASLEAIMGLESSSELFTISVNGVLYLQMGQWTSVFLDNA
SGCSLTVRSGSHFSAVLLGV

As used herein, the term “SF3B1” denotes a gene which encodes subunit 1 of the splicing factor 3b protein complex. Splicing factor 3b, together with splicing factor 3a and a 12S RNA unit, forms the U2 small nuclear ribonucleoproteins complex (U2 snRNP). The splicing factor 3b/3a complex binds pre-mRNA upstream of the intron's branch site in a sequence independent manner and may anchor the U2 snRNP to the pre-mRNA. Splicing factor 3b is also a component of the minor U12-type spliceosome. The carboxy-terminal two-thirds of subunit 1 have 22 non-identical, tandem HEAT repeats that form rod-like, helical structures. Alternative splicing results in multiple transcript variants encoding different isoforms. The sequence of said gene can be found under the Ensembl accession number ENSG00000115524.

According to the invention, the mutations in SF3B1 can be the SF3B1^{E622D}, the SF3B1^{Y623C}, the SF3B1^{R625L}, the SF3B1^{R625C}, the SF3B1^{R625H}, the SF3B1^{N626Y}, the SF3B1^{N626D}, the SF3B1^{H662D}, the SF3B1^{H662Q}, the SF3B1^{K666M}, the SF3B1^{K666E}, the SF3B1^{K666Q}, the SF3B1^{K666N}, the SFR3B1^{K666R}, the SF3B1^{K666T}, the SF3B1^{K700E}, the SF3B1^{I704N}, the SF3B1^{I704F}, the SF3B1^{K742D}, the SF3B1^{G742D}, the SF3B1^{D781G} and the SF3B1^{D781E}.

According to the invention, a treatment of anemia according to the invention can be an erythropoiesis-stimulating agents including recombinant erythropoietin, biosimilars, Immunomodulatory imide drugs (ImiDs) like lenalidomide, activin IIB receptor agonists like luspatercept and other TGF-β family member ligand traps, selective serotonin reuptake inhibitors (SSRIs), proline hydroxylase inhibitors emethylating agents like azacitidine or low doses of iron chelator deferasirox.

According to the invention, a treatment of iron overload can be an iron chelator like Desferal (DCI deferoxamine), Exjade (DCI deferasirox), Ferriprox (deferriprone) or other siderophores like desferrithiocin or synthetic chelators molecules potentially used in other indications like clioquinol in the treatment of neuro-degeneration, dexrazoxane a cardioprotective agent, triapine an anticancer therapy, floranol a treatment against atherosclerosis or phytic acid used in cardiovascular diseases. Other drugs, currently in development for the treatment of iron overload, can be used like recombinant BMP6, RNAi to Tmprss6, hepcidin agonists like mini-hepcidins which are small drug-like hepcidin agonists, or drugs interfering with erythroid regulator of hepcidin, ERFE.

As used herein, the terms “determining, in a sample obtained from the patient, the expression of a variant of the transcript or the protein of ERFE” denotes the detection a variant of the transcript or of the protein of ERFE by variety of techniques. In other word, the simple detection of a variant of the transcript or of the protein of ERFE even in a little amount, will allow to diagnose or monitor an anemia in a patient suffering from a myeloid malignancies with at least one mutation in the SF3B1 gene. In a particular embodiment, the expression (and notably the expression level) of the variant ERFE⁺¹² or of the variant ERFE^{VPFQ} can be done. In another particular embodiment, a fragment of the ERFE^{VPFQ} can be detected like the peptide VPFQFGLPGPPGPPGPQGPPGPIIPPEALLK of SEQ ID NO: 7.

Methods for measuring variant of the protein ERFE (the level of the variant) in a biological sample may be assessed by any of a wide variety of well-known methods from one of skill in the art for measuring the level of a polypeptide including, but not limited to, direct methods like mass spectrometry-based quantification methods, with or without prior fractionation techniques such as HPLC or other type of chromatography, protein microarray methods, enzyme immunoassay (EIA), radioimmunoassay (MA), Western blot analysis, Mesoscale discovery (MSD), Luminex, ELISPOT and Enzyme Linked Immunoabsorbant Assay (ELISA).

Said direct analysis can be assessed by contacting the biological sample with a binding partner capable of selectively interacting with the polypeptide present in the biological sample. The binding partner may be an antibody that may be polyclonal or monoclonal, preferably monoclonal (e.g., a isotope-labelled, element-labelled, radio-labelled, chromophore-labelled, fluorophore-labelled, or enzyme-labelled antibody), an antibody derivative (e.g., an antibody conjugate with a substrate or with the protein or ligand of a protein of a protein/ligand pair (e.g., biotin-streptavidin), or an antibody fragment (e.g., a single-chain antibody, an isolated antibody hypervariable domain, etc.) which binds specifically to the polypeptide. In some embodiments, the binding partner may be the antibody of the invention. In another embodiment, the binding partner may be the aptamer of the invention.

The binding partners of the invention such as antibodies or aptamers, may be labelled with a detectable molecule or substance, such as an isotope, a chemical element, a fluorescent molecule, a radioactive molecule, an enzyme or any others labels known in the art. Labels are known in the art that generally provide (either directly or indirectly) a signal.

As used herein, the term “labelled”, with regard to the antibody, is intended to encompass direct labelling of the antibody or aptamer by coupling (i.e., physically linking) a detectable substance, such as an isotope, an element, a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance. An antibody or aptamer of the invention may be produced with a specific isotope or a radioactive molecule by any method known in the art. For example radioactive molecules include but are not limited to radioactive atom for scintigraphic studies and positron emission tomography (PET) such as I123, I124, In111, Re186, Re188, specific isotopes include but are not limited to 13C, 15N, 126I, 79Br, 81Br.

The aforementioned assays generally involve the binding of the binding partner (ie. antibody or aptamer) to a solid support. Solid supports which can be used in the practice of the invention include an ELISA plate, an ELIspot plate, a bead (e.g., a cytometric bead, a magnetic bead), a microarray (e.g., a SIMS microarray), a slide or a plate. Said supports may e.g., be coated with substrates such as nitrocellulose (e. g., in glass, membrane or microtiter well form); polyvinylchloride (e. g., sheets or microtiter wells); polystyrene latex (e.g., beads or microtiter plates); polyvinylidene fluoride; diazotized paper; nylon membranes; activated beads, magnetically responsive beads, silicon wafers.

In a particular embodiment, an ELISA method can be used, wherein the wells of a microtiter plate are coated with a set of antibodies which recognize said polypeptide. A biological sample containing or suspected of containing said polypeptides is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art such as Singulex, Quanterix, MSD, Bioscale, Cytof.

In one embodiment, an Enzyme-linked immunospot (ELISpot) method may be used. Typically, the biological sample is transferred to a plate which has been coated with the desired anti-polypeptide capture antibodies. Revelation is carried out with biotinylated secondary Abs and standard colorimetric or fluorimetric detection methods such as streptavidin-alkaline phosphatase and NBT-BCIP and the spots counted.

In one embodiment, when multi-polypeptide level measurement is required, use of beads bearing binding partners of interest may be preferred. In a particular embodiment, the bead may be a cytometric bead for use in flow cytometry. Such beads may for example correspond to BD™ Cytometric Beads commercialized by BD Biosciences (San Jose, Calif.) or LUMINEX® beads or ERENNA® (SINGULEX®) beads. Typically cytometric beads may be suitable for preparing a multiplexed bead assay. A multiplexed bead assay, such as, for example, the BD™ Cytometric Bead Array, is a series of spectrally discrete beads that can be used to capture and quantify soluble antigens. Typically, beads are labelled with one or more spectrally distinct fluorescent dyes, and detection is carried out using a multiplicity of photodetectors, one for each distinct dye to be detected. A number of methods of making and using sets of distinguishable beads have been described in the literature. These include beads distinguishable by size, wherein each size bead is coated with a different target-specific antibody (see e.g. Fulwyler and McHugh, 1990, Methods in Cell Biology 33:613-629), beads with two or more fluorescent dyes at varying concentrations, wherein the beads are identified by the levels of fluorescence dyes (see e.g. European Patent No. 0 126,450), and beads distinguishably labelled with two different dyes, wherein the beads are identified by separately measuring the fluorescence intensity of each of the dyes (see e.g. U.S. Pat. Nos. 4,499,052 and 4,717,655). Both one-dimensional and two-dimensional arrays for the simultaneous analysis of multiple antigens by flow cytometry are available commercially. Examples of one-dimensional arrays of singly dyed beads distinguishable by the level of fluorescence intensity include the BD™ Cytometric Bead Array (CBA) (BD Biosciences, San Jose, Calif.) and Cyto-Plex™ Flow Cytometry microspheres (Duke Scientific, Palo Alto, Calif.). An example of a two-dimensional array of beads distinguishable by a combination of fluorescence intensity (five levels) and size (two sizes) is the QuantumPlex™ microspheres (Bangs Laboratories, Fisher, Ind.). Another example is the SIMOA™ technology (QUANTERIX™). An example of a two-dimensional array of doubly-dyed beads distinguishable by the levels of fluorescence of each of the two dyes is described in Fulton et al. (1997, Clinical Chemistry 43(9):1749-1756). The beads may be labelled with any fluorescent compound known in the art such as e.g. FITC (FL1), PE (FL2), fluorophores for use in the blue laser (e.g. PerCP, PE-Cy7, PE-Cy5, FL3 and APC or Cy5, FL4), fluorophores for use in the red, violet or UV laser (e.g. Pacific blue, pacific orange). In another particular embodiment, bead is a magnetic bead for use in magnetic separation. Magnetic beads are known to those of skill in the art. Typically, the magnetic bead is preferably made of a magnetic material selected from the group consisting of metals (e.g. ferrum, cobalt and nickel), an alloy thereof and an oxide thereof. In another particular embodiment, bead is bead that is dyed and magnetized.

In another particular embodiment, beads are labelled with an isotope or a (chemical) element, and beads are identified by elemental analysis in a mass spectrometer (Cytof).

In one embodiment, protein microarray methods may be used. Typically, at least one antibody or aptamer directed against the polypeptide(s) is immobilized or grafted to an array(s), a solid or semi-solid surface(s). A biological sample containing or suspected of containing the polypeptide(s) is then labelled with at least one isotope or one element or a reactive tag or one fluorophore or one colorimetric tag that are not naturally contained in the tested biological sample. After a period of incubation of said biological sample with the array sufficient to allow the formation of antibody-antigen complexes, the array is then washed and dried. After all, quantifying said polypeptides may be achieved using any appropriate microarray scanner like fluorescence scanner, colorimetric scanner, SIMS (secondary ions mass spectrometry) scanner, MALDI scanner, electromagnetic scanner, electrochemoluminescent scanner or any technique allowing to quantify said labels. In another embodiment, the antibody or aptamer grafted on the array is labelled.

In one embodiment, a mass spectrometry-based quantification methods may be used. Mass spectrometry-based quantification methods may be performed using either labelled or unlabelled approaches [DeSouza and Siu, 2012]. Mass spectrometry-based quantification methods may be performed using chemical labelling, metabolic labelling or proteolytic labelling. Mass spectrometry-based quantification methods may be performed using mass spectrometry label free quantification, a quantification based on extracted ion chromatogram (EIC) and then profile alignment to determine differential level of polypeptides.

In one embodiment, a mass spectrometry-based quantification method particularly useful can be the use of targeted mass spectrometry methods as selected reaction monitoring (SRM), multiple reaction monitoring (MRM), parallel reaction monitoring (PRM), data independent acquisition (DIA) and sequential window acquisition of all theoretical mass spectra (SWATH) [Moving target Zeliadt N 2014 The Scientist; Liebler Zimmerman Biochemistry 2013 targeted quantitation pf proteins by mass spectrometry; Gallien Domon 2015 Detection and quantification of proteins in clinical samples using high resolution mass spectrometry. Methods v81 p 15-23; Sajic, Liu, Aebersold, 2015 Using data-independent, high-resolution mass spectrometry in protein biomarker research: perspectives and clinical applications. Proteomics Clin Appl v9 p 307-21].

In one embodiment, the mass spectrometry-based quantification is used to do peptide and/or protein profiling can be use with matrix-assisted laser desorption/ionisation time of flight (MALDI-TOF), surface-enhanced laser desorption/ionization time of flight (SELDI-TOF; CLINPROT) and MALDI Biotyper apparatus [Solassol, Jacot, Lhermitte, Boulle, Maudelonde, Mangé 2006 Clinical proteomics and mass spectrometry profiling for cancer detection. Journal: Expert Review of Proteomics V3, 13, p 311-320; FDA K130831].

In one embodiment, ELISA sandwich specifically designed to measure the polypeptide of the invention may be used. The principle is a sandwich ELISA with a capturing antibody against the C-terminus and the second antibody is against the N-terminus. This sandwich ELISA gives the concentration of the polypeptide.

In a particular embodiment, to assess the production of variant ERFE, the inventors have started the setting of a serum dosage using LC MS/MS (liquid chromatography-mass spectrometry). For this purpose, they have defined the parameters of the specific VPFQ peptide (SEQ ID NO: 5) generated by trypsin digestion of the recombinant variant protein ERFE^{VPFQ} (SE ID NO:4). Then they were able to target this peptide among the serum protein after depletion of the most abundant proteins including albumin.

Measuring the expression (and notably the expression level) of a variant of the transcript of ERFE can be performed by a variety of techniques well known in the art.

Typically, the expression of a transcript (and notably the expression level) may be determined by determining the quantity of mRNA. Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labelled nucleic acid molecule provides an indicator of the presence or concentration of a target nucleic acid sequence (e.g., genomic target nucleic acid sequence) (to which the labelled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antllranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6diclllorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281: 20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can he produced that are identifiable based on their different spectral characteristics. For example, semiconductor nanocrystals can he produced that emit light of different colors based on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlsbad, Calif.).

Additional labels include, for example, radioisotopes (such as 3 H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hybridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labelled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labelled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 0.1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labelled with fluorophores (including fluorescent dyes and QUANTUM DOTS®) can be directly optically detected when performing FISH. Alternatively, the probe can be labelled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, coumarin-based compounds, Podophyllotoxin, Podophyllotoxin-based compounds, and combinations thereof), ligand or other indirectly detectable moiety. Probes labelled with such non-fluorescent molecules (and the target nucleic acid sequences to which they bind) can then be detected by contacting the sample (e.g., the cell or tissue sample to which the probe is bound) with a labelled detection reagent, such as an antibody (or receptor, or other specific binding partner) specific for the chosen hapten or ligand. The detection reagent can be labelled with a fluorophore (e.g., QUANTUM DOT®) or with another indirectly detectable moiety, or can be contacted with one or more additional specific binding agents (e.g., secondary or specific antibodies), which can be labelled with a fluorophore.

In other examples, the probe, or specific binding agent (such as an antibody, e.g., a primary antibody, receptor or other binding agent) is labelled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/0117153.

It will be appreciated by those of skill in the art that by appropriately selecting labelled probe-specific binding agent pairs, multiplex detection schemes can he produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can he labelled with a first hapten, such as biotin, while a second probe that corresponds to a second target sequence can be labelled with a second hapten, such as DNP. Following exposure of the sample to the probes, the bound probes can he detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can he added to the multiplex detection scheme using other spectrally distinct fluorophores. Numerous variations of direct, and indirect (one step, two step or more) can he envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and subjecting the RNAs to amplification and hybridization to specific probes, more particularly by means of a quantitative or semi-quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In another embodiment, the expression level is determined by metabolic imaging (see for example Yamashita T et al., Hepatology 2014, 60:1674-1685 or Ueno A et al., Journal of hepatology 2014, 61:1080-1087).

According to all methods of the invention, the detection of the transcript variant of ERFE can be done by specific RT-qPCR or fluorescent PCR analyzed by capillary electrophoresis and the detection of the protein variant of ERFE can be done by detection of the peptide VPFQ (SEQ ID NO: 5) alone or in the protein ERFE of SEQ ID NO:3 (like the detection of the protein ERFE^{VPFQ} of SEQ ID NO:4 or of the peptide of SEQ ID NO:7).

Predetermined reference values used for comparison may comprise “cut-off” or “threshold” values that may be determined as described herein. Each reference (“cut-off”) value for the transcript/protein′ expression may be predetermined by carrying out a method comprising the steps of

a) providing a collection of samples from patients suffering of a patient suffering from a myeloid malignancy with at least one mutation in the SF3B1 gene or anemia with myeloid malignancies with at least one mutation in the SF3B1 gene (after diagnosis of these diseases for example);

b) determining the expression level of a variant (transcript or protein) for each sample contained in the collection provided at step a);

c) ranking the tissue samples according to said expression level of the variants and determining a threshold value above which the expression level is said to be “high” and below which the expression level is said to be “low”;

d) quantitatively defining the threshold/cut-off/reference value by determining the number of copies of the said gene corresponding to the threshold/cut-off/reference value; to be done by constructing a calibration curve using known input quantities of cDNA or protein for the said variants;

e) classifying said samples in pairs of subsets of increasing, respectively decreasing, number of members ranked according to their expression level,

f) providing, for each sample provided at step a), information relating to the actual clinical outcome for the corresponding cancer patient (i.e. the diagnosis, the response for a treatment;

g) for each pair of subsets of samples, obtaining a Kaplan Meier percentage of diagnosis/response curve;

h) for each pair of subsets of samples calculating the statistical significance (p value) between both subsets

i) selecting as reference value for the expression level, the value of expression level for which the p value is the smallest.

For example the expression level of the variants (transcript or protein) has been assessed for 100 samples from 100 patients. The 100 samples are ranked according to their expression level. Sample 1 has the highest expression level and sample 100 has the lowest expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding patient, Kaplan Meier curves are prepared for each of the 99 groups of two subsets. Also for each of the 99 groups, the p value between both subsets was calculated.

The reference value is selected such as the discrimination based on the criterion of the minimum p value is the strongest. In other terms, the expression level corresponding to the boundary between both subsets for which the p value is minimum is considered as the reference value. It should be noted that the reference value is not necessarily the median value of expression levels.

In routine work, the reference value (cut-off value) may be used in the present method to discriminate samples and therefore the corresponding patients.

Kaplan-Meier curves of percentage of survival as a function of time are commonly used to measure the fraction of patients living for a certain amount of time after treatment and are well known by the person skilled in the art.

The man skilled in the art also understands that the same technique of assessment of the expression level of a variants (transcript or protein) should of course be used for obtaining the reference value and thereafter for assessment of the expression level of a gene of a patient subjected to the method of the invention.

Such predetermined reference values of expression level may be determined for any variants defined above.

In another particular embodiment, a step of communicating the result to the patient may be added to all the methods of the invention. The result can be a result about the diagnostic of anemia or systemic iron overload or the results of the monitoring of anemia and systemic iron overload.

In another embodiment, the invention relates to a method for detecting a variant of the ERFE transcript or a variant of the ERFE protein in a sample of a patient suffering from a myeloid malignancy by a technique allowing the detection of a transcript variant of protein variant as listed above.

In another aspect, the invention relates to a method for detecting a variant of the ERFE transcript or a variant of the ERFE protein and/or evaluating its amount in a biological sample.

In another embodiment, when a diagnostic of an anemia with at least one mutation in the SF3B1 gene or of a systemic iron overload is done according to the methods of the invention or when the monitoring of an anemia or of a systemic iron overload according to the methods of the invention reveal that the treatment does not work, a new treatment can be administrated to the patient to treat the anemia with at least one mutation in the SF3B1 gene or to treat the systemic iron overload. Such treatment are listed above in the description.

Thus, the invention also relates to a method of treatment of anemia and/or systemic iron overload in a patient with at least one mutation in the SF3B1 gene and which have be diagnosed as having an anemia with at least one mutation in the SF3B1 gene or a systemic iron overload comprising administrating to said patient a treatment of anemia or iron overload as listed above.

### Therapeutic Methods

Considering that elevated level of ERFE accounts for hyperferritinemia that precedes any transfusion in SF3B1-mutated MDS, decreasing the ERFE level could be therapeutic option to reduce the extend of hyperferritinemia and prevent iron overload and toxicity. Because both wildtype and variant ERFE proteins are produced, targeting the aberrant variant isoform fits the aim of reducing the amount of ERFE without abrogating the regulation of hepcidin and daily iron metabolism by the normal isoform.

For this purpose, the inventors have designed two types of antisens oligonucleotides: LNA-ASO from Exiqon (Qiagen) and morpholino-ASO from GeneTools. Their specificity toward ERFE⁺¹² and inhibitory efficiency are being tested in human cell lines engineered to express SF3B1 mutation using CRISPR-Cas9.

Thus, the invention also relates to an inhibitor of a variant of the transcript or protein of ERFE for use in the treatment of an anemia and/or of a systemic iron overload in a patient suffering from a myeloid malignancy with at least one mutation in the SF3B1 gene.

Thus, the invention also relates to an isolated, synthetic or recombinant oligonucleotide recognizing or targeting a variant of the transcript of ERFE for use in the treatment of an anemia and/or of a systemic iron overload in a patient suffering from a myeloid malignancy with at least one mutation in the SF3B1 gene.

In a particular embodiment, the isolated, synthetic or recombinant oligonucleotide can be used to reduce the expression level of ERFE transcript and subsequently of the protein, to reduce the repression of hepcidin and limit the level of circulating plasma iron and reduce the risk of secondary hemochromatosis.

The term “oligonucleotide” also refers to an oligonucleotide sequence that is inverted relative to its normal orientation for transcription and so correspond to a RNA or DNA sequence that is complementary to a target gene mRNA molecule expressed within the host cell (e.g., it can hybridize to the target gene mRNA molecule through Watson-Crick base pairing). An antisense strand may be constructed in a number of different ways, provided that it is capable of interfering with the expression of a target gene. For example, the antisense strand can be constructed by reverse-complementing the coding region (or a portion thereof) of the target gene relative to its normal orientation for transcription to allow the transcription of its complement, (e.g., RNAs encoded by the antisense and sense gene may be complementary). In some embodiments, the oligonucleotide need not have the same intron or exon pattern as the target gene, and noncoding segments of the target gene may be equally effective in achieving antisense suppression of target gene expression as coding segments such as antisense oligonucleotide (ASO). In some embodiments, the oligonucleotide have the same exon pattern as the target gene such as siRNA and antisense oligonucleotide (ASO).

According to the invention, the antisense oligonucleotide of the present invention targets an mRNA encoding a variant of ERFE, and is capable of reducing the amount of a variant of ERFE in cells. As used herein, an oligonucleotide that “targets” an mRNA refers to an oligonucleotide that is capable of specifically binding to said mRNA. That is to say, the antisense oligonucleotide comprises a sequence that is at least partially complementary, particularly perfectly complementary, to a region of the sequence of said mRNA, said complementarity being sufficient to yield specific binding under intra-cellular conditions. As immediately apparent to the skilled in the art, by a sequence that is “perfectly complementary to” a second sequence is meant the reverse complement counterpart of the second sequence, either under the form of a DNA molecule or under the form of a RNA molecule. A sequence is “partially complementary to” a second sequence if there are one or more mismatches. The antisense oligonucleotide of the present invention that target an mRNA encoding a variant of ERFE may be designed by using the sequence of said mRNA as a basis, e.g. using bioinformatic tools. For example, the sequence of SEQ ID NO: 2 (sequence of the gene variant of ERFE) can be used as a basis for designing nucleic acids that target an mRNA encoding a variant of ERFE. Particularly, the antisense oligonucleotide according to the invention is capable of reducing the amount of a variant of ERFE in cells. Methods for determining whether an oligonucleotide is capable of reducing the amount of a variant of ERFE in cells are known to the skilled in the art. This may for example be done by analyzing a variant of ERFE RNA expression such as by RT-qPCR, in situ hybridization or a variant of ERFE protein expression such as by immunohistochemistry, Western blot, and by comparing a variant of ERFE protein expression in the presence and in the absence of the antisense oligonucleotide to be tested.

In a particular embodiment the oligonucleotide recognizes or targets the variant of SEQ ID NO: 2.

In a particular embodiment, the ASO can have the following sequences:

(SEQ ID NO: 8)
AACTGAAAGGGAAC,
(SEQ ID NO: 9)
AAAGGGAACCTTGGCAGTGAGGACA
or
(SEQ ID NO: 10)
ACCTTGGCAGTGAGGACATGT.

In a particular embodiment, the ASO can have 90%, 91%, 92%, 93%, 94%, 95%, 96%, 97%, 98% or 99% of homology with the SEQ ID NO: 8, 9 or 10.

The invention also relates to ASOs having the following sequences:

(SEQ ID NO: 8)
AACTGAAAGGGAAC,
(SEQ ID NO: 9)
AAAGGGAACCTTGGCAGTGAGGACA
or
(SEQ ID NO: 10)
ACCTTGGCAGTGAGGACATGT.

In a particular embodiment, an endonuclease can be used to reduce or abolish the expression of the gene, transcript or protein variants of ERFE.

Indeed, as an alternative to more conventional approaches, such as cDNA overexpression or downregulation by RNA interference, new technologies provide the means to manipulate the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the error prone non homologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671), zebrafish (Hwang et al., 2013, PLoS One, Vol. 8:e68708), C. elegans (Hai et al., 2014 Cell Res. doi: 10.1038/cr.2014.11), bacteria (Fabre et al., 2014, PLoS Negl. Trop. Dis., Vol. 8:e2671), plants (Mali et al., 2013, Science, Vol. 339: 823-826), Xenopus tropicalis (Guo et al., 2014, Development, Vol. 141: 707-714), yeast (DiCarlo et al., 2013, Nucleic Acids Res., Vol. 41: 4336-4343), Drosophila (Gratz et al., 2014 Genetics, doi:10.1534/genetics.113.160713), monkeys (Niu et al., 2014, Cell, Vol. 156: 836-843), rabbits (Yang et al., 2014, J. Mol. Cell Biol., Vol. 6: 97-99), pigs (Hai et al., 2014, Cell Res. doi: 10.1038/cr.2014.11), rats (Ma et al., 2014, Cell Res., Vol. 24: 122-125.) and mice (Mashiko et al., 2014, Dev. Growth Differ. Vol. 56: 122-129). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In a particular embodiment, the invention relates to a method for treating MDS, MDS-RS or anaemia by administrating to a patient in need thereof an isolated, synthetic or recombinant oligonucleotide recognizing or targeting a variant of the gene or transcript of ERFE.

In another embodiment, an oligonucleotide can be used to correct the aberrant splicing which results in the apparition of the variant of ERFE transcript.

Thus, the invention also relates to an isolated, synthetic or recombinant oligonucleotide recognizing or targeting the wild type gene ERFE for us in the treatment an anemia and/or of an systemic iron overload in a patient suffering from a myeloid malignancy with at least one mutation in the SF3B1 gene.

The term “oligonucleotide” also refers to an oligonucleotide sequence that is inverted relative to its normal orientation for transcription and so correspond to a RNA or DNA sequence that is complementary to a target gene mRNA molecule expressed within the host cell (e.g., it can hybridize to the target gene mRNA molecule through Watson-Crick base pairing). An antisense strand may be constructed in a number of different ways to increase the wild type ERFE transcript and protein levels or restore by splicing-modulation the transcription of the wild type ERFE transcripts encoding a totally or partially functional protein. For example, the antisense strand can be constructed by reverse complementing the coding region (or a portion thereof) of the target gene relative to its normal orientation for transcription (e.g., RNAs encoded by the antisense and sense gene may be complementary). In some embodiment, the antisense oligonucleotide strand need not have the same intron or exon pattern as the target gene, and noncoding segments of the target gene. In some embodiment, antisense oligonucleotide is a target-site blocker (TSB) (such as miRNA binding-blocker oligonculeotides (MBBO)) or a splicing-blocker oligonucleotides (SBO).

According to the invention, the antisense oligonucleotide of the present invention targets an mRNA encoding the wild type ERFE, and is capable of increasing the wild type ERFE transcript and protein levels or restoring by splicing-modulation the transcription of the wild type ERFE transcripts encoding a totally or partially functional protein in cells. As used herein, an oligonucleotide that “targets” an mRNA refers to an oligonucleotide that is capable of specifically binding to said mRNA. That is to say, the antisense oligonucleotide comprises a sequence that is at least partially complementary, particularly perfectly complementary, to a region of the sequence of said mRNA, said complementarity being sufficient to yield specific binding under intra-cellular conditions. As immediately apparent to the skilled in the art, by a sequence that is “perfectly complementary to” a second sequence is meant the reverse complement counterpart of the second sequence, either under the form of a DNA molecule or under the form of a RNA molecule. A sequence is “partially complementary to” a second sequence if there are one or more mismatches. The antisense oligonucleotide of the present invention that target an mRNA encoding the wild type ERFE may be designed by using the sequence of said mRNA as a basis, e.g. using bioinformatic tools. For example, the sequence of SEQ ID NO: 1 can be used as a basis for designing nucleic acids that target an mRNA encoding the wild type ERFE. Particularly, the antisense oligonucleotide according to the invention is capable of increasing the amount of the wild type ERFE. Methods for determining whether an oligonucleotide is capable of increasing the amount of the wild type ERFE in cells are known to the skilled in the art. This may for example be done by analyzing the wild type ERFE transcript expression such as by PCR, RT-qPCR, in situ hybridization or the wild type ERFE protein expression such as by immunohistochemistry, Western blot, and by comparing the wild type ERFE transcript expression or the wild type ERFE protein expression in the presence and in the absence of the antisense oligonucleotide to be tested.

The invention also relates to a primer that specifically binds to a variant of the transcript of ERFE. Particularly, the primer binds to the variant of SEQ ID NO: 2.

In a particular embodiment, the invention relates to a primer that specifically binds to a variant of the transcript ERFE comprising the nucleic acids sequence SEQ ID NO: 6.

According to the invention, any primer of the invention will be used to amplify a variant of the transcript ERFE in techniques using such tools like a PCR.

In a particular embodiment, an antibody against a variant of the protein ERFE like ERFE^{VPFQ} can be used for the treatment of an anemia and/or of a systemic iron overload in a patient suffering from a myeloid malignancy with at least one mutation in the SF3B1 gene.

According to the present invention, “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated VL-CDR1, VL-CDR2, VL-CDR3 and VH-CDR1, VH-CDR2, VH-CDR3, respectively. An antigen-binding site, therefore, includes six CDRs, comprising the CDR set from each of a heavy and a light chain V region. Framework Regions (FRs) refer to amino acid sequences interposed between CDRs.

Antibodies directed against a protein variant of ERFE like ERFE^{VPFQ} can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against a protein variant of ERFE like ERFE^{VPFQ} can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce anti-DHODH or Chk1 single chain antibodies. Compounds useful in practicing the present invention also include anti-protein variant of ERFE like ERFE^{VPFQ} antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to a protein variant of ERFE like ERFE^{VPF}.

Humanized anti-protein variant of ERFE like ERFE^{VPFQ} antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

In another embodiment, the antibody according to the invention is a single domain antibody against a protein variant of ERFE like ERFE^{VPFQ}. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In one embodiment, the compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

The invention also relates to an antibody that specifically binds to a variant of the protein of ERFE. Particularly, the antibody binds to the variant of SEQ ID NO: 4.

In a particular embodiment, the invention relates to an antibody that specifically binds to a variant of the protein ERFE comprising the amino acids sequence SEQ ID NO: 5.

In a particular embodiment, the invention relates to an antibody that specifically binds to a peptide of SEQ ID NO: 7.

In a particular embodiment, the invention relates to an antibody that specifically binds to a variant of the protein ERFE comprising the amino acids sequence SEQ ID NO: 7.

### Therapeutic Composition

Another object of the invention relates to a therapeutic composition comprising an antibody which specifically binds to a variant of the protein ERFE or an ASO according to the invention for use in the treatment of an anemia and/or of an iron overload in a patient suffering from a myeloid malignancy.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Pharmaceutical compositions of the present invention may comprise a further therapeutic active agent. The present invention also relates to a kit comprising an antibody and/or an ASO according to the invention and a further therapeutic active agent.

The invention will be further illustrated by the following figures and examples. However, these examples and figures should not be interpreted in any way as limiting the scope of the present invention.

### FIGURES

FIG. 1: Identification of ERFE^{VPFQ} peptide by mass spectrometry and hepcidin repression by recombinant variant ERFE^{VPFQ} protein. ERFE^{VPFQ} is a potent suppressor of hepcidin. Recombinant human ERFE^{WT} and ERFE^{VPFQ} were produced in 293F cells. Hep3B hepatoma cells were treated with supernatants of 293F cells overexpressing ERFE^{WT} and ERFE^{VPFQ} for 16 hours. HAMP was quantified by RT-qPCR and its expression was normalized to HPRT quantities. Data shown are means±SEM of four independent experiments and represent a fold change of hepcidin mRNA expression in ERFE treated compared to untreated (CTRL) cells. Two-tailed Student t-test for P-values.

FIG. 2: Increased plasma concentration of ERFE in SF3B1^{MUT} MDS patients. Dosages were performed in plasma collected from 20 non-blood donor healthy volunteers, 156 MDS patients including 94 SF3B1^{MUT} and 62 SF3B1^{WT} representing the learning cohort (A-D) and from 55 MDS patients including 42 SF3B1^{MUT} and 13 SF3B1^{WT} representing the validation cohort (E-H). Quantification of erythroferrone (A, E), ferritin (B, F), hepcidin (C, G) and hepcidin/ferritin ratio (D, H). Results are expressed as medians±interquartile ranges (IQR). The boxplots represent the median and the first and third quartiles and the whiskers represent the lowest value still within the 1.5 IQR of the lower quartile and the highest value still within the 1.5 IQR of the upper quartile. Mann-Whitney for P-values.

FIG. 3: Biological parameters of 156 MDS patients of the learning cohort. (A) Serum EPO level according to the concentration of ERFE. Pearson test for correlation; P=0.403. (B) Comparison of soluble transferrin receptor (sTfR) level between 94 SF3B1^{MuT}, 61 SF3B1^{WT} and 20 healthy non-blood donor controls. Results are expressed as medians±IQR. Mann-Whitney test for P-values. ns: not significant.

FIG. 4: Biological parameters of low transfusion burden MDS patients of the learning cohort. Comparison of ferritin (A), plasma iron (B), hepcidin/ferritin ratio (C), hepcidin/plasma iron ratio (D) and ERFE (E) between 25 SF3B1^{MuT} and 36 SF3B1^{MUT} patients. Results are expressed as medians±IQR. Mann-Whitney test for P-values.

FIG. 5: ERFE⁺¹² expression is a biomarker of clonal erythropoiesis. (A) Fluorescent PCR was performed in 14 paired samples from SF3B1^{MUT} MDS patients enrolled in the GFM-LenEpo-08 clinical trial (8 non-responding and 6 responding patients). Three examples of each category are shown. Peak heights of ERFE⁺¹² and ERFE^{WT} signals were integrated as ERFE⁺¹²/ERFE⁺¹²+ERFE^{WT} ratios at screening and evaluation after 4 cycles of treatment. Ratios are indicated. (B) Evolution of the ERFE⁺¹²/ERFE⁺¹²+ERFE^{WT} ratio between screening and evaluation at 4 cycles of treatment in 8 non-responding patients (left) and 6 responding patients (right). (C) Percent variation of ERFE⁺¹²/ERFE⁺¹²+ERFE^{WT} ratios in 8 responding and 6 non-responding patients (left). Percent variation of SF3B1 variant allele frequency (VAF) available for 6/8 non-responding and 4/6 responding patients is also shown (right). Results are expressed as medians±interquartile ranges (first and third quartiles). Mann-Whitney test for P-values.

TABLE 1
Clinical and biological characteristics of the learning cohort of 156 MDS
patients according to SFB1 status.

| | Learning cohort | | | |
|---|---|---|---|---|
| | All | SF3B1^{WT} | SF3B1^{MUT} | |
| Parameters | n = 156 | n = 62 | n = 94 | P-value |
| Age | | | | |
| Median, years (range) | 74 (46-91) | 74 (54-88) | 74 ( 46-91) | 0.371 |
| Sex | | | | |
| male, no. (%) | 92 (59) | 38 (61) | 54 (57) | 0.740 |
| WHO-Subtype 2016 | | | | |
| 5q/MDS-SLD no. (%) | 19 (12.2) | 17 (27.4) | 2 (2.1) | <0.0001 |
| MDS-SLD-RS/MDS- | 77 (49.4) | 7 (11.3) | 70 (74.5) | |
| MLD-RS, no. (%) | | | | |
| MDS-MLD, no. (%) | 42 (26.9) | 25 (40.3) | 17 (18.1) | |
| MDS-EB1, no. (%) | 18 (11.5) | 13 (21.0) | 5 (5.3) | |
| Hemoglobin (G/L) | | | | |
| Median (IQR) | 8.6 | 9.2 | 8.2 | <0.001 |
| | (7.8-9.4) | (8.3-9.7) | (7.7-9.0) | |
| Neutrophil count (G/L) | | | | |
| Median (IQR) | 2.6 | 2.2 | 2.8 | 0.502 |
| | (1.6-3.7) | (1.3-3.7) | (1.6-3.8) | |
| Platelet count (G/L) | | | | |
| Median (IQR) | 234 | 172 | 255 | 0.008 |
| | (164-325) | (103-308) | (195-329) | |
| Karyotype IPSS | | | | |
| Good, no. (%) | 125 (80.2) | 48 (77.4) | 77 (81.9) | 0.133 |
| Intermediate, no. (%) | 23 (14.7) | 10 (16.1) | 13 (13.8) | |
| Poor, no. (%) | 3 (1.9) | 3 (4.9) | 0 (0.0) | |
| Unavailable, no. (%) | 5 (3.2) | 1 (1.6) | 4 (4.3) | |
| Karyotype IPSS-R | | | | |
| Very good, no. (%) | 7 (4.5) | 4 (6.4) | 3 (3.2) | 0.270 |
| Good, no. (%) | 122 (78.2) | 46 (74.2) | 76 (80.9) | |
| Intermediate, no. (%) | 20 (12.8) | 9 (14.5) | 11 (11.7) | |
| Poor, no. (%) | 2 (1.3) | 2 (3.2) | 0 (0.0) | |
| Unavailable, no. (%) | 5 (3.2) | 1 (1.6) | 4 (4.2) | |
| BM blasts (%) | | | | |
| Median (IQR) | 2.0 | 2.0 | 2.0 | 0.267 |
| | (1.0-3.5) | (1.0-4.0) | (1.0-3.0) | |
| BM erythroblasts (%) | | | | |
| Median (IQR) | 25 (11-38) | 15 (3-27) | 32 (19-45) | <0.0001 |
| IPSS | | | | |
| Low risk, no. (%) | 85 (54.5) | 27 (43.6) | 58 (61.7) | 0.082 |
| Int-1, no. (%) | 64 (41.0) | 32 (51.6) | 32 (34.0) | |
| Int-2, no. (%) | 1 (0.6) | 1 (1.6) | 0 (0.0) | |
| Unavailable, no. (%) | 6 (3.9) | 2 (3.2) | 4 (4.3) | |
| IPSS-R | | | | |
| Very low risk, no. (%) | 15 (9.6) | 9 (14.5) | 6 (6.4) | 0.107 |
| Low, no. (%) | 95 (60.9) | 31 (50.0) | 64 (68.1) | |
| Int, no. (%) | 36 (23.1) | 17 (27.4) | 19 (20.2) | |
| High, no. (%) | 4 (2.6) | 3 (4.9) | 1 (1.1) | |
| Unavailable, no. (%) | 6 (3.8) | 2 (3.2) | 4 (4.2) | |
| Serum EPO level (U/L) | | | | |
| Median (IQR) | 99 (39-249) | 84 | 101 | 0.657 |
| | | (30-200) | (47-312) | |
| Red blood | | | | |
| cells unit, no | | | | |
| Median (range) | 4 (0-17) | 2 (0-15) | 5.5 (0-17) | <0.0001 |

TABLE 2
Clinical and biological characteristics of the validation cohort of
55 MDS patients according to SFB1 status

| | Validation cohort | | | |
|---|---|---|---|---|
| | All | SF3B1^{WT} | SF3B1^{MUT} | |
| Parameters | n = 55 | n = 13 | n = 42 | P-value |
| Age | | | | |
| Median, years (range) | 74 (28-94) | 76 (28-94) | 74 (30-90) | 0.374 |
| Sex | | | | |
| male, no. (%) | 31 (56) | 8 (62) | 23 (55) | 0.750 |
| WHO-Subtype 2016 | | | | |
| 5q/MDS-SLD, no. (%) | 4 (7.3) | 4 (30.8) | 0 (0.0) | <0.0001 |
| MDS-SLD-RS/ | 41 (74.5) | 4 (30.8) | 37 (88.1) | |
| MDS-MLD- | | | | |
| RS/MDSMPN-RS-T, | | | | |
| no. (%) | | | | |
| MDS-MLD, no. (%) | 6 (10.9) | 3 (23.1) | 3 (7.1) | |
| MDS-EB1, no. (%) | 4 (7.3) | 2 (15.3) | 2 (4.8) | |
| Hemoglobin (G/L) | | | | |
| Median (IQR) | 8.0 | 7.8 | 8.1 | 0.097 |
| | (6.2-8.8) | (5.5-8.2) | (7.3-9.0) | |
| Neutrophil count (G/L) | | | | |
| Median (IQR) | 3.0 | 3.3 | 2.7 | 0.435 |
| | (1.8-3.8) | (2.1-3.9) | (1.6-3.8) | |
| Platelet count (G/L) | | | | |
| Median (IQR) | 293 | 232 | 298 | 0.561 |
| | (222-388) | (113-399) | (227-381) | |
| Karyotype IPSS | | | | |
| Good, no. (%) | 46 (83.6) | 10 (76.9) | 36 (85,7) | 0.031 |
| Intermediate, no. (%) | 5 (9.2) | 0 (0) | 5 (11.9) | |
| Poor, no. (%) | 2 (3.6) | 2 (15.4) | 0 (0) | |
| Unavailable, no. (%) | 2 (3.6) | 1 (7.7) | 1 (2.4) | |
| Karyotype IPSS-R | | | | |
| Very good, no. (%) | 2 (3.6) | 1 (7.7) | 1 (2.4) | 0.535 |
| Good, no. (%) | 45 (81.8) | 9 (69.2) | 36 (85.7) | |
| Intermediate, no. (%) | 6 (11.0) | 2 (15.4) | 4 (9.5) | |
| Unavailable, no. (%) | 2 (3.6) | 1 (7.7) | 1 (2.4) | |
| BM blasts (%) | | | | |
| Median (IQR) | 2.0 | 3.0 | 2.0 | 0.070 |
| | (1.0-3.0) | (1.8-4.0) | (1.0-3.0) | |
| BM erythroblasts (%) | | | | |
| Median (IQR) | 34 (26-42) | 36 (16-42) | 34 (27-44) | 0.788 |
| IPSS | | | | |
| Low risk, no. (%) | 37 (67.3) | 7 (53.8) | 30 (71.4) | 0.419 |
| Int-1, no. (%) | 16 (29.1) | 5 (38.5) | 11 (26.2) | |
| Unavailable, no. (%) | 2 (3.6) | 1 (7.7) | 1 (2.4) | |
| IPSS-R | | | | |
| Very low risk, no. (%) | 8 (14.6) | 1 (7.7) | 7 (16.7) | 0.106 |
| Low, no. (%) | 32 (58.2) | 5 (38.5) | 27 (64.2) | |
| Int, no. (%) | 13 (23.6) | 6 (46.1) | 7 (16.7) | |
| Unavailable, no. (%) | 2 (3.6) | 1 (7.7) | 1 (2.4) | |
| Red blood cells unit, no | | | | |
| Median (range) | 4 (0-9) | 4 (0-8) | 4 (0-9) | 0.879 |

### EXAMPLE

Material & Methods

Patients

For the learning cohort, lower risk MDS patients (n=156) were recorded between 2008 and 2017. Bone marrow (BM) aspirates and peripheral blood (PB) plasmas were collected after each patient gave his informed consent for biological investigations according to the recommendations of institutional review board and local ethics committee (Institutional review board (IRB) numbers: IdF X GFM-LenEpo-08 EudraCT 2008-008262-12; IdFII 2010-A00033-36; IdFV 212-A01395-38 EudraCT 2012-002990-7338; OncoCCH 2015-08-11-DC). For the validation cohort, lower risk MDS patients (n=55) were recorded prospectively after patient gave his consent for plasma collection in France (5 centres; IRB Onco-CCH 2015-08-11DC) and Germany (one centre; IRB Ethikkommission an der TU Dresden; EK 115032015) between 2016 and 2018. BM samples from 10 age-matched controls and PB plasmas from 20 healthy controls were also collected. BM mononuclear cells were purified on Ficoll gradient. Cell pellets were processed for DNA extraction and further put in Trizol (Thermo Fisher Scientific, Waltham, Mass.) before RNA extraction. Low transfusion burden was defined as <4 RBC units per 8 weeks.

Genomic Studies

BM mononuclear cells were purified on Ficoll gradient and were processed for DNA extraction using the DNA/RNA Kit (Qiagen, Hilden, Germany). Mutations in a selected panel of 26 genes (ASXL1, CBL, DNMT3A, ETV6, EZH2, FLT3, IDH1, IDH2, JAK2, KIT, KRAS, NRAS, MPL, NPM1, PHF6, PTPN11, RIT1, RUNX1, SETBP1, SF3B1, SRSF2, TET2, TP53, U2AF1, WT1 and ZRSR2) were screened by a Next-Generation Sequencing (NGS) assay using the Ion AmpliSeg™ library kit2 384 (Life Technologies, Chicago, Ill.). Multiplex PCR amplifications (233 primer pairs) were performed from 2×10 ng of genomic DNA. After amplification, barcodes and adaptors were added to amplicons by ligation. Products were subjected to a selective purification on AMPure beads (Life Technologies). Emulsion PCR (emPCR) was performed using the OneTouchV2 (Life Technologies) instrument. Sequencing was performed on Ion PGM™ (Life Technologies) onto the 318 V2 chip (15 samples per chip). All the samples were also screened for ASXL1 (including c.1934dupG; p.G646WfsX12) and SRSF2 mutations by Sanger sequencing. JAK2, NPM1, and FLT3-ITD mutations were also investigated by qPCR and fluorescent PCR. For analysis, base calls were generated by the Torrent Browser software using the included variant caller with an additional plug-in (Life Technologies). The .bam and .vcf files were used for further analysis. The .vcf files were annotated with the Ion reporter software (Life Technologies) and processed for a second analysis of the indexed files using the NextGENe software (Softgenetics, State College, Pa.). Results were compared to select abnormalities that will be further considered.

RNA-Sequencing

cDNA synthesis was conducted with MuLV Reverse Transcriptase (Invitrogen, Carlsbad, Calif), with quality controls conducted on an Agilent 2100 Bioanalyzer (Agilent, Les Ulis, France). Libraries were constructed using the TruSeq Stranded mRNA Sample Preparation Kit (Illumina, San Diego, Calif.) and sequenced on an Illumina HiSeq 2500 platform using a 100-bp paired-end sequencing strategy. An average depth of global sequence coverage of 114 million and a median coverage of 112 million was attained. For analysis, TopHat (v2.0.6) was used to align the reads against the human reference genome Hg19 RefSeq (RNA sequences, GRCh37) downloaded from the UCSC Genome Browser (http://genome.ucsc.edu).

Bioinformatic Analysis of RNA-Sequencing

FASTQ files were mapped using TopHat (v2.0.6)28 to align the reads against the human reference genome Hg19 RefSeq (RNA sequences, GRCh37) downloaded from the UCSC Genome Browser (http://genome.ucsc.edu). Read count normalizations and groups comparisons were performed using DESeq2, which converts the counts to log-counts per million with associated precision weights and flags were computed using a custom algorithm within R (The R project for Statistical Computing [http://www.r-project.org/]). Assuming that a maximum of 80% of genes are expressed we selected the 20% lowest counts for each sample as background. A threshold is fixed at two standard deviations over the mean of the background. All transcripts for which normalized counts were lower than the computed threshold were designated as background for each array. When comparing normalized counts between two groups, a transcript was included in the analysis if its counts exceeded the background in at least 80% of the sample from at least 1 group. To identify differentially expressed transcripts, we used empirical Bayes estimation. For differential expression study, the results obtained after Deseq2 comparison were selected for further analysis and filtered at P-value≤0.05 and fold-changes of 1.2, 1.5 or 2. Principal component analysis calculated from averaged RefSeq exons robust multiarray average (RMA) values by RefSeq gene symbol was generated using R and the GGplot2 package. The bivariate normal density is a function of the means and standard deviations of the X and Y variables, PC 1 and PC2. The ellipsoid was computed from the bivariate normal distribution fit to each group. Read counts for splicing junctions from junctions. bed TopHat output were considered. Differential analysis was performed on junction read counts using DESeq2 (29). Only alternative acceptor splice sites (two or more 30ss with junctions to the same 50ss) and alternative donor splice sites (two or more 50ss with junctions to the same 30ss) were considered for this analysis. According to Alsafadi et al., predictions and analysis of branch point sequence analysis were done with the online tools SVM-BPfinder (http://regulatorygenomics.upf.edu/Software/SVM_BP/) and the Human Splicing Finder (http://www.umd.be/HSF/) (13, 57, 58). The SVM_BP code was altered to allow for BP six base pairs from the 3′ss by setting minidist3ss=6 in svm_getfeat.py.

Fluorescent PCR and Quantitative PCR

RNA was extracted using RNeasy Mini Kit (Qiagen) and used as a template for cDNA synthesis with the Maxima First Strand cDNA Synthesis Kit (Thermo Fisher Scientific). cDNA was then amplified by PCR (Phire Hot Start II DNA Polymerase, Thermo Fisher Scientific) using specific 5′FAM-forward or 5′FAM-reverse primers. Finally, DNA fragment analysis was performed by capillary electrophoresis: 1 μl of diluted PCR product was added to 0.2 μl of GeneScan 500 ROX dye Size Standard (Thermo Fisher Scientific) and 18 μl of RNAse-free water. The DNA was denatured 5 minutes at 95° C. and placed at 4° C. Fragments were separated using a 3730×1 DNA Analyzer and data were analyzed using GeneMapper Software 5 (Thermo Fisher Scientific). For RT-qPCR, the mean amplification efficiency of each PCR was 95%. According to the MIQE guidelines, the expression of each gene of interest was normalized on the expression of two housekeeping genes B2M and ACTB.

Transfection

UT-7/EPO cell line was transfected with a synthetic full length SF3B1^{WT} or mutant SF3B1^{K700E} cDNA provided by Dr S. Buonamici (H3 Biomedicine Inc, Cambridge, Mass.) using the Amaxa program T-024 in R solution (Lonza, Basel, Switzerland).

CRISPR/Cas9 generation of isogenic SF3B1^{K700E} and SF3B1^{WT} cell lines

The murine erythroid cell line G1E-ER4 was used to generate isogenic SF3B1^{K700E} and SF3B1^{WT} cell lines using CRISPR/Cas9-stimulated homology-mediated repair (59). Cells were transfected using Amaxa Nucleofector™ 2b Device program A-024 and Nucleofector kit L (Lonza) with Cas9 (Ref. 41815, Addgene, Cambridge, Mass), a mSf3b1-specific gRNA (built from gRNA cloning vector, Ref 41824, Addgene) and a donor template encoding a hygromycin selection cassette at a 1:1:1 ratio. After two days, hygromycin was added to the culture at 800 μg/mL for 72 h, and maintained at 400 μg/mL for another 72 h. Hygromycin-resistant cells were then single cell-sorted using the FACS Aria III (Becton Dickinson Biosciences, Franklin Lakes, N.J.) and clones were analyzed 3 weeks later by DNA sequencing to assess the presence of the SF3B1^{K700E} mutation. The selection cassette was removed by flippase-mediated excision. SF3B1^{K700E} and SF3B1^{WT} hygromycin-resistant clones were transfected with a flippase-encoding vector and single cell-sorted. Cassette-excised clones were assessed by PCR and SF3B1^{K700E} and SF3B1^{WT} expression was verified by RT-PCR and cDNA Sanger sequencing.

ERFE and ENOSF1 Minigenes

The ERFE minigene was synthesized from the linearized pET01 Exontrap vector (Mobitec, Göttingen, Germany) in which we insert an ERFE sequence centered on alternative 3′ss (AG′) flanked upstream of 100 bp in intron 2-3 and downstream of 100 bp in exon 3. The ERFE insert was PCR-amplified from the genomic DNA of MOLM-14 cells using Phusion High-Fidelity DNA Polymerase 2 U/μl (Thermo Fisher Scientific), and purified with the QIAquick PCR Purification Kit (Qiagen). We introduced 16 bp of homology with the ends of the linearized vector at 5′-end of the forward and reverse primers. Using In-Fusion HD Cloning Kit (Clontech, Mountain View, Calif), we cloned the purified amplicon between the XhoI and XbaI restriction sites of Exontrap vector containing a functional splice donor site. A validated ENOSF1 minigene was used as a control (13). G1E-ER4 9.2 (SF3B1^{WT}) and G1E-ER4 5.13H (SF3B1^{K700E}) cells were transfected by electroporation (Amaxa Nucleofector™ 2 b, Lonza) with 400 ng of ERFE or ENOSF1 minigene in L solution. After 24 hours, total RNA was extracted with RNeasy Mini Kit (Qiagen) and processed for fluorescent PCR.

Primary Cell Culture

For erythroid and granulocytic cell expansion, CD34⁺ cells were isolated from the mononuclear cell fraction of bone marrow samples using the MidiMacs system (Miltenyi Biotec, Bergisch Gladbach, Germany). For erythroid differentiation, CD34⁺ cells, which purity was higher than 80%, were cultured at 0.8×10⁶ per mL for 4 days in Iscove's modification of Dulbecco medium containing 15% BIT9500 (Miltenyi Biotech), penicillin/streptomycin, L-Glutamine, Epo 2 UI/mL (Roche, Basel, Switzerland), SCF 100 ng/mL (Miltenyi Biotech), IL6 10 ng/mL (Miltenyi Biotech) and dexamethasone 2.10⁻⁷ M (Sigma Merck, Darmstadt, Germany). Cells were diluted every day in the same medium until day 4. From day 4, IL6 was removed. From days 10 to 16 cells were switched to Epo (2 UI/mL) to obtain terminal erythroid differentiation followed by flow cytometry (GPA, CD71, CD49d and Band3). For granulocytic differentiation, CD34⁺ cells were cultured in the same medium with SCF 100 ng/mL, IL3 10 ng/mL (Miltenyi Biotech) and G-CSF 20 ng/mL (Sandoz, Holzkirchen, Germany). Every 2 days, MGG staining and RT-qPCR for erythroid genes (GATA1, HBB) or granulocytic genes (MPO, SPI1) were performed.

Cell Sorting

Cells were sorted using CD45-PE, CD3-FITC, CD19-ECD and CD5-AA700 antibodies (Beckman Coulter, Brea, USA) from the mononuclear cell fraction of bone marrow or blood samples using FACS ARIA3 device (Becton Dickinson, Franklin Lakes, USA). In sorted cell populations, RNA was extracted and SF3B1 gene was sequenced by Sanger method.

Mass Spectrometry Analysis

Sequencing grade trypsin (50 ng) purchased from Promega was used to digest proteins of erythroblast lysates in solution at 37° C. overnight. Resulting products were analyzed by nano liquid chromatography hyphenated to a Q-Exactive Plus mass spectrometer (Thermo) operating in data dependent scheme: peptides loaded on the chromatography were trapped and washed with 0.1% TFA 2% Acetonitrile in milliQH₂O on a C18 reverse phase precolumn (Acclaim Pepmap 100 Angstroms pores, 5 μm particles, 2 cm long, 75 μm inner diameter) for 3 minutes at 5 μL/min. Trapped peptides were then separated on a C18 reverse phase analytical column (2 μm particle size, 100 Å pore size, 75 μm inner diameter, 25 cm length) with a 1.5 hours gradient starting from 99% of solvent A containing 0.1% formic acid in milliQH₂O and ending in 40% of solvent B containing 80% ACN and 0.085% formic acid in milliQH₂O. The mass spectrometer acquired data throughout the elution process. The MS scans spanned from 350 to 1500 Th with AGC target 1.10⁶ with 60 ms MIIT and resolution of 70 000. HCD fragmentations were performed on the 10 most abundant ions with a dynamic exclusion time of 30 s. Precursor selection window was set at 2Th. HCD Normalized Collision Energy (NCE) was set at 27% and MS/MS scan resolution was set at 17,500 with AGC target 1.10⁵ within 60 ms MIIT. Spectra were recorded in profile mode. The mass spectrometry data were analyzed using Mascot 2.5.1 (www.matrixscience.com) The database used was a concatenation of human sequences from the Uniprot-Swissprot database (release 2017-10, 20314 sequences) and a customized database of modified sequences including the ERFE^{VPFQ} sequence, and a list of in-house common contaminant sequences. Cystein carbamidomethylation was set as constant modification, methionine oxidation and proline hydroxylation were set as variable modifications. Spectrum annotation was done according to nomenclature of peptide fragmentation proposed by Roepstorff and Fohlman (60).

Recombinant ERFE^{WT} and ERFE⁺¹² Production and Purification

Human ERFE^{WT} and ERFE⁺¹² cDNA sequences were cloned into pcDNA3.1 with the following modifications: vector signal sequence (interleukin-2) was used instead of the native, followed by a spacer and a FLAG tag. Recombinant proteins were produced in suspension culture in Freestyle 293F cells (Life Technologies) transiently transfected using Freestyle F-MAX reagent (Life Technologies). Supernatants from cells overexpressing FLAG-tagged ERFE proteins were collected after 5 days and supplemented with protease inhibitor cocktail. Recombinant proteins were purified using an anti-FLAG affinity gel according to the manufacturer's protocol (Sigma-Aldrich Chimie, Saint-Quentin-Fallavier, France) and eluted with 150 μg/mL FLAG peptide (Sigma). FLAG peptide was eliminated by filtering preparations through an Amicon Ultra 30K device (Merck Millipore, Guyancourt, France), and recombinant ERFE proteins were suspended in a saline solution (0.9% NaCl). Protein concentration was determined using Coomassie Imperial Protein Stain and Pierce bicinchoninic acid protein assay (Thermo Fisher Scientific).

Western Blot Analysis

Supernatants from 293F cells secreting ERFE^{WT} or ERFE^{VPFQ} cells were subjected to SDS-PAGE in reducing (diluted in Laemmli buffer 10% 2-mercaptoethanol and incubated for 5 minutes at 95° C.) and non-reducing (diluted in Laemmli buffer without 2-mercaptoethanol) conditions and electroblotted to nitrocellulose membranes (Biorad). Membranes were blocked with 5% of dry milk in TBS-T buffer (10 mM Tris-HCl, pH 7.5, 150 mM NaCl, 0.15% Tween 20) and incubated 2 h at RT with an anti-DDK monoclonal antibody conjugated to horseradish peroxidase (HRP) (86861, Cell Signaling), which recognizes DYKDDDDK epitope (FLAG-Tag) or with rabbit polyclonal antibodies to ERFE (produced by YenZym) overnight at 4° C. before incubation with a goat anti-rabbit HRP-linked antibody (7074S, Cell signaling). Enzyme activity was visualized by an ECL-based detection system (GE Lifesciences). Blot imaging and analysis was performed on a Chemidoc MP Imaging System (Bio-Rad) with the Image Lab software.

Hepcidin Repression in Hep3B Hepatoma Cell Line

Hep3B hepatoma cell line were incubated overnight (16 hours) in D-MEM Glutamax (Life Technologies) supplemented with 10% fetal bovine serum and 50% (v/v) supernatants from control freestyle 293-F cells or 293F cells transfected with a plasmid encoding ERIE^{WT} or ERFE⁺¹² cDNA. Total RNA from Hep3B cells was extracted using UPzol Total RNA Isolation Reagent (Biotech Rabbit). cDNA was synthesized using MMLV-RT (Promega). Quantitative PCR (qPCR) reactions were prepared with LightCycler® 480 DNA SYBR Green I Master reaction mix (Roche Diagnostics) and run in duplicate on a LightCycler® 480 System (Roche Diagnostics). HAMP mRNA transcript abundance was normalized to the reference gene HPRT.

Human Hepcidin and Soluble Transferrin Receptor Dosages

Plasma soluble transferrin receptor assays were performed on Dimension VISTA 1500 (Siemens Healthcare, Saint Denis, France). Circulating hepcidin was quantified from EDTA-plasma samples by the previously described liquid chromatography coupled with tandem mass spectrometry (LC-MS/MS) method (61).

Human Erythroferrone Immunoassay

Plasma erythroferrone concentration was determined as previously described²⁸.

Briefly, high binding 96-well plates (Corning) were coated with overnight at 4° C. with 100 μL/well of 1.0 μg/mL capture antibody in 50 mM sodium carbonate buffer (pH 9.6). Plates were washed (TBS, 0.5% Tween-20) and blocked for 1 h at RT with 200 μL/well blocking buffer (PBS, 0.2% Na casein, 0.05% Tween 20, 0.1M NaCl). Recombinant human ERFE standard was serially diluted to 10, 5, 2.5, 1.25, and 0.625 ng/mL. After a 1-hour incubation at room temperature, the plate was washed and incubated for 1 hour with 100 mL per well biotinylated rabbit monoclonal antibody to human erythroferrone (1 μg/mL). The plate was then washed, incubated for 45 minutes with Neutravidin-horseradish peroxidase conjugate (ThermoScientific #31030) 1/5000 (100 mL/well), washed 3 times and developed with 100 μL TMB Substrate System for ELISA (ThermoScientific #34028) at RT in the dark for 10 min. The reaction was stopped by adding 50 μl of 2N sulfuric acid and plates were read on a Spectramax 250 (Molecular Devices) at 450 nm.

Statistical Analysis

For quantitative variables, values were expressed as median and interquartile range (IQR) or means and standard error of the mean (SEM) and compared using the Mann-Whitney test. Categorical variables reported as counts or percentages were compared using Chi-square or Fisher's exact tests. For transcript quantification, the Mann-Whitney test was used to assign a statistical significance for each group comparison. P-values <0.05 were considered significant. A multivariate logistic regression analysis was adjusted for selected variables chosen with a P-value <0.1 in univariate analysis (JMP version 10.0.2, SAS Institute Inc, Cary, N.C.).

Results:

Upregulation of ERFE by the Use of an Alternative 3′ Splice Site in SF3B1-Mutated MDS

To investigate the mechanism of systemic iron overload, a cohort of 156 patients with MDS including 60 MDS-RS with single lineage dysplasia (SLD), 17 MDS-RS with multilineage dysplasia (MLD), 2 5q-syndrome, 17 MDS-SLD, 42 MDS-MLD, and 18 MDS with type 1 excess of blasts (EB1) was established (table 1). The Revised-International Prognosis Scoring System score was very low in 21, low in 98, intermediate in 29, and high in 2 patients. Twenty-six genes commonly mutated in MDS were sequenced in the BM mononuclear cell (MNC) fraction. SF3B1 gene was mutated in 94 MDS including 63 (67%) cases affected by a SF3B1^{K700E} mutation. Among the 62 patients with no SF3B1 mutation, other splicing genes, SRSF2, U2AF1 or ZRSR2 were mutated in 27 cases, and no splicing gene mutation was observed in 35 cases. Some cases presented two splicing mutations (data not shown). We evaluated the consequences of SF3B1 gene mutations on gene expression and splicing by sequencing the transcriptome of the BM MNCs isolated from 27 patients of the cohort including 21 SF3B1^{MUT} MDS, 6 SF3B1^{WT} MDS, and 5 healthy controls. Differential analyses of gene expression and splice junctions were conducted using DESeq2 (29). We detected 6,343 genes as differentially expressed between SF3B1^{MUT} MDS and SF3B1^{WT} MDS with a P-value <0.05 (data not shown). Principal component analysis (PCA) of gene expression profiles separated SF3B1^{MUT} and SF3B1^{WT} MDS (data not shown). The differentially expressed genes were enriched in 73 specific GO terms with an absolute log₂ fold-change (FC) >1 and a Benjamini-Hochberg (BH)-adjusted P-value <0.05 (data not shown). Genes with a log₂(FC)<−1 were involved in serine/threonine kinase signaling, apoptosis, myeloid differentiation, inflammation, cell-cell adhesion while those with a log₂(FC) >1 were involved in heme metabolism, erythrocyte differentiation, and iron homeostasis (data not shown). We plotted the log₂(FC) of 16 differentially expressed genes belonging to the IRON_ION_HOMEOSTASIS gene set GO:0055072 (http://amigo.geneontology.org) and showed that the FAM132B/ERFE transcript encoding erythroferrone was up-regulated (data not shown).

We then identified 1,528 differentially expressed 5′ and 3′ junctions including annotated 5′ donor and 3′ acceptor ss, ambiguous junctions and canonical junctions with BH-adjusted P-values ≤10⁻⁵ and absolute log₂(FC)≥1 (data not shown). These junctions allowed the hierarchical clustering of the 21 SF3B1^{MUT} and 6 SF3B1^{WT} MDS samples (data not shown). After excluding differentially expressed canonical junctions, we then considered the 1,147 alternative junctions among which we identified 786 3′ acceptor junctions (68.5%), 176 5′ donor junctions (15.3%) and 185 ambiguous junctions (16.1%) attributed to either the alternative 5′ or 3′ss or both alternative 5′ and 3′ss (data not shown). The analysis of distances between the alternative and canonical 3′ss showed that the majority of alternative 3′ss (AG′) was located between −24 and −9 nucleotides preceding the canonical 3′ss (AG) (data not shown). The proportion of alternative AG′ junctions in these novel splice variants was generally increased in SF3B1 mutants and less frequently in SF3B1^{WT} samples (data not shown). To detect alternative transcripts that were likely to generate significant amounts of variant proteins with a modified length, we applied a filter selecting transcripts with an additional stretch of nucleotides numbering multiples of 3, a percentage of alternative junction coverage (AG′) to alternative and canonical junctions coverage (AG′+AG) over 0.1 and an expression ratio of the alternative junction in SF3B1^{MUT} versus SF3B1^{WT} samples over 10 (data not shown). We obtained 66 alternative junctions in 63 genes (data not shown). These genes were involved in the epigenetic regulation and transcription, mRNA processing and translation, intracellular transport, cell cycle and migration, signaling and apoptosis, mitochondrial metabolism and iron homeostasis (data not shown). Among the 66 alternative junctions, 29 were related to an in-frame insertion of 9 to 27 nucleotides and 26 of them were differentially expressed between SF3B1^{MUT} and SF3B1^{WT} MDS with an absolute log₂(FC) >0.3 (data not shown). FAM132B/ERFE gene was identified among the upregulated genes with an alternative junction due to the use of a cryptic 3′ss located between the exons 2 and 3 (chr2: 239,070,357-239,071,364) and no PTC. The aberrant transcript contained 12 additional nucleotides in the open reading frame and is referred to as ERFE⁺¹² from here on (data not shown). It was systematically detected in all SF3B1^{MUT} MDS samples and represented a mean of 24.8% of FAM132B/ERFE transcripts in SF3B1^{MUT} MDS versus 0.2% in SF3B1^{WT} MDS. This indicates that the expression of ERFE⁺¹² was related to the presence of a mutation in SF3B1 gene.

SF3B1^{MUT}-Restricted Expression of Alternative ERFE⁺¹² Transcript

To ascertain that expression of the ERFE⁺¹² transcript is dependent on the presence of a SF3B1 mutation, we transfected the erythro-megakaryocytic cell line UT7/EPO with a pLVX plasmid encoding a synthetic full-length SF3B1^{WT} or mutant SF3B1^{K700E} cDNA. We then designed a sensitive fluorescent RT-PCR allowing the detection of ERFE⁺¹² and ERFE^{WT} transcripts as 162 nucleotides (nt) and 150 nt fragments by capillary electrophoresis (data not shown). Twenty-four hours post-transfection, ERFE⁺¹² transcript was detected in SF3B1^{K700E} transfected cell line, but not in SF3B1^{WT} cell line (data not shown). To validate the splice pattern induced in the context of SF3B1 mutation in ERFE pre-mRNA, we performed a minigene splicing assay. An ERFE sequence of about 200 nucleotides located on both sides of the cryptic 3′ss (AG′) was cloned in an ExonTrap vector. The alternative junction in ENOSF1 gene (chr18: 683,395-685,920) cloned in the same vector was used as a control (13). These minigenes were transfected into the murine G1E-ER4 cell line in which the SF3B1^{K700E} mutation was edited by CRISPR-Cas9 technology. As expected due to the lack of intron homology between species, sequencing the transcriptome of G1E-ER4 SF3B1^{K700E} cell line or isogenic SF3B1^{WT} cell line demonstrated that endogenous murine ERFE and ENOSF1 did not undergo alternative splicing (not shown). After transfection, the alternative 3′ss AG′ ERFE⁺¹² was detected on the capillary electrophoresis in G1E-ER4 SF3B1^{K700E} cells, but not in the isogenic G1E-ER4 SF3B1^{WT} cells (data not shown). The usage of alternative 3′ss AG′ was detectable for ENOSF1 gene in SF3B1^{WT} cell line and became predominant in SF3B1^{K700E} cell line suggesting that the mutation favored the usage of alternative AG′.

We then investigated the expression of ERFE⁺¹² in primary BM samples of MDS patients using the fluorescent PCR. In 46 lower risk MDS patients, SF3B1^{MUT} was present in 25 cases including 20 MDS-SLD-RS/MDS-MLD-RS, 3 MDS-MLD, and 2 MDS-EB1. ERFE⁺¹² was detected in the BM MNC fractions of all SF3B1^{MUT} MDS, and none of the SF3B1^{WT} MDS. ERFE⁺¹² was not detected in any other cases of MDS with mutations in SRSF2 (n=10), U2AF I (n=1), ZRSR2 (n=1), or in 21 MDS patients with mutations in other genes (data not shown). In one patient with an SF3B1 monoallelic deletion and a substitution G742D on the remaining allele, the expression of alternative ERFE⁺¹² exceeded that of the canonical ERFE^{WT} (data not shown). We then amplified primary erythroblasts from the BM CD34⁺ cells of two SF3B1^{MUT} and one SF3B1^{WT} MDS and showed that ERFE⁺¹² was expressed in SF3B1^{MUT} but not in SF3B1^{WT} erythroid cells (data not shown). This further supports that ERFE⁺¹² is related to SF3B1 mutation. SF3B1^{uT} MDS are characterized by the enrichment of the BM with erythroid cells. To investigate whether ERFE⁺¹² could be detected in cells deriving from erythroblastic BMs with another genetic background, we collected samples from one patient with an acquired sideroblastic anemia with 38% of BM erythroblasts, more than 15% of ring sideroblasts, a normal karyotype, no SF3B1 mutation but one SRSF2 and one TET2 mutation, three patients with a congenital sideroblastic anemia due to an ALAS2 mutation/deletion or a GLRX5 mutation, one patient with a severe β-thalassemia and one patient with SF3B1^{K700E} MDS. Using fluorescent PCR, ERFE^{WT} was present while ERFE⁺¹² was not detectable in any case except the SF3B1^{K700E} MDS sample (data not shown). This confirms that the onset of an aberrant ERFE⁺¹² is not dependent on the amplification of erythroid compartment or the presence of ring sideroblasts, but depends on the presence of a mutant SF3B1.

Translation of ERFE⁺¹² into a Variant Protein that Represses Hepcidin

Human ERFE encodes a 354-aminoacid polypeptide. The addition of 12 nucleotides in ERFE mRNA generates an ERFE variant (further referred to as ERFE^{VPFQ}) containing a valine-proline-phenylalanine-glutamine (VPFQ) insertion immediately upstream of the collagen domain (data not shown). To investigate whether the mutant ERFE^{VPFQ} protein was produced in vivo, we prepared cell lysates of erythroblasts derived in culture from the BM CD34⁺ cells of patients with SF3B1^{MUT} MDS. Through LC MS/MS protein identification, we obtained several peptide-matching propositions including the ALHELGVYYLPDAEGAFR peptide (SEQ ID NO: 11) (data not shown) already reported in public databases (www.proteomicsdb.org/) and we identified a cryptic peptide VPFQFGLPGPPGPPGPQGPPGPIIPPEALLK (SEQ ID NO: 7) corresponding to the VPFQ insertion at position 108 of ERFE (data not shown). This confirms that alternative ERFE⁺¹² transcript is translated into ERFE^{VPFQ} in SF3B1^{MUT} erythroblasts.

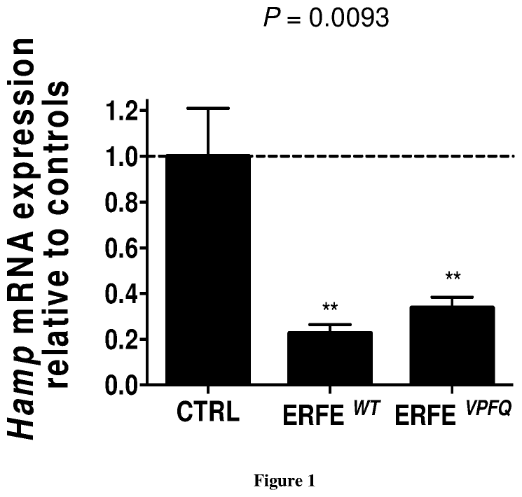

ERFE is known to repress hepcidin in mice and contribute to pathological hepcidin suppression in patients with transfused and non-transfused β-thalassemia (27, 28). Whether ERFE^{VPFQ} also repressed hepcidin was tested. For this purpose, recombinant proteins ERFE^{WT} and ERFE^{VPFQ} were produced in HEK293F cells. SDS-PAGE analysis of ERFE^{WT} and ERFE^{VPFQ} in reducing and non-reducing conditions demonstrated a comparable molecular weight and a multimerization pattern as both proteins were predominantly found in a trimeric form of approximately 130 kDa (data not shown). Then, Hep3B hepatoma cells were treated for 16 hours with HEK293F cell supernatants overexpressing ERFE^{VPFQ} or ERFE^{WT}. Both proteins caused a similar 5-fold reduction in HAMP mRNA expression compared to controls (FIG. 1). These data indicate that insertion of 4 amino-acids upstream of the collagen domain did not affect the bioactivity of the protein.

Overproduction of Erythroferrone is Predictive of Hyperferritinemia in SF3B1^{MUT} MDS

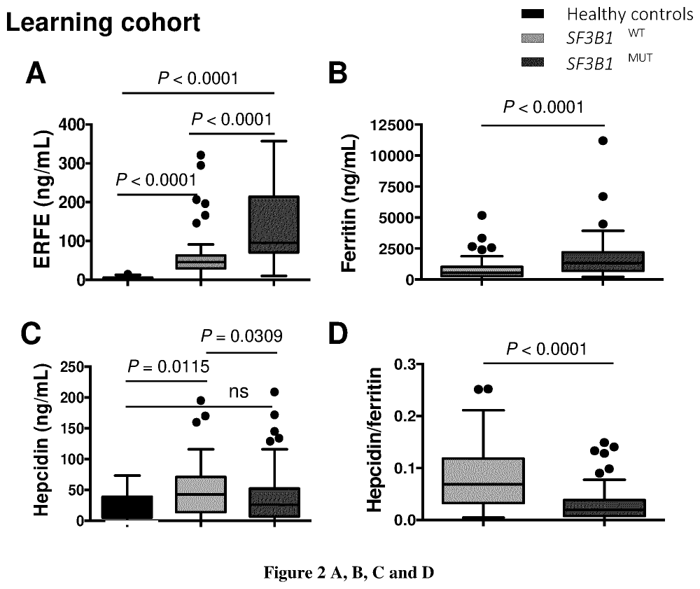

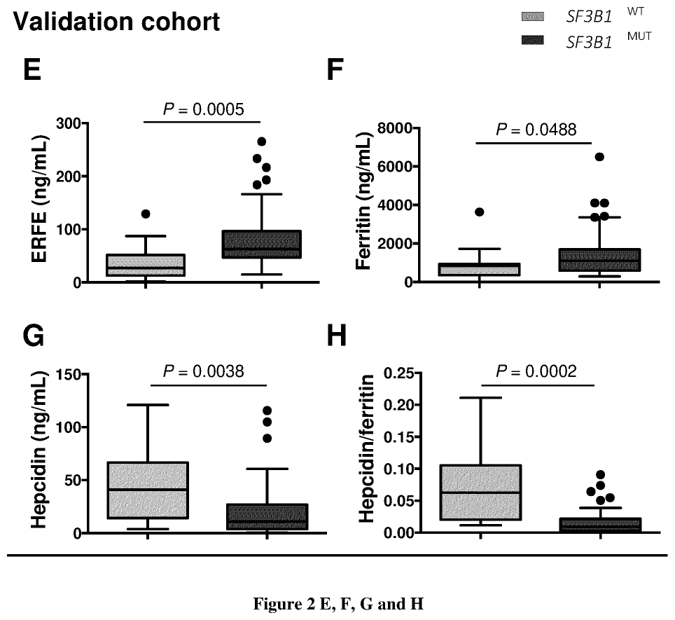

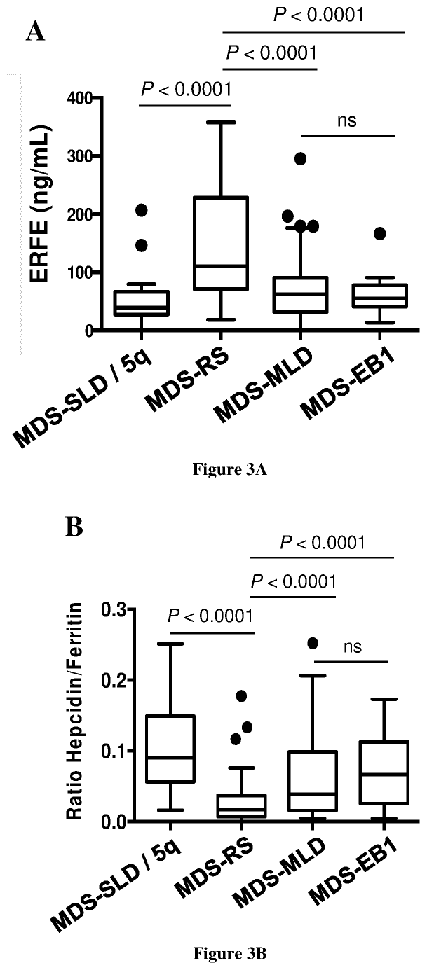

We then measured the level of plasma ERFE in the learning cohort of 156 MDS patients and 20 healthy non-blood donor controls using a validated immunoassay (28). We first verified that the ERFE immunoassay was able to detect both ERFE^{WT} and ERFE^{VPFQ}. Indeed, human ERFE ELISA detected similar amounts (1 μg/mL) of each recombinant protein in the supernatants of HEK293F cells transiently transfected with ERFE^{WT} and ERFE^{VPFQ} expression vectors. This establishes that both isoforms were detectable by ELISA. The mean concentration of ERFE in SF3B1^{MUT} or SF3B1^{WT} MDS was higher than in controls (P<0.0001; FIG. 2A). Among the MDS samples, the ERFE concentration was higher in SF3B1^{MUT} (135.0±72.5 ng/mL) compared to SF3B1^{WT} (62.1±36.7 ng/mL) MDS (P<0.0001; FIG. 2A). Consistently, the ERFE concentration was also higher in MDS-RS compared to all other WHO MDS subtypes (data not shown). We then measured plasma hepcidin levels and confirmed that the concentration of hepcidin in SF3B1^{MUT} MDS was significantly lower compared to SF3B1^{WT} MDS (P=0.0309; FIG. 2B). We found no difference between SF3B1^{MUT} MDS and healthy controls. Ferritin levels were significantly higher in SF3B1^{MUT} patients compared to SF3B1^{WT} patients (P<0.0001; FIG. 2C). The hepcidin/ferritin ratio was even more decreased in SF3B1^{MUT} versus SF3B1^{WT} MDS (P<0.0001; FIG. 2D) and also in MDS-RS compared to other WHO subtypes (data not shown), and this was due to both a lower level of hepcidin and a higher concentration of ferritin in SF3B1^{MUT} patients. The plasma concentration of ERFE was inversely correlated to the hepcidin/ferritin ratio (Pearson test; P<0.0001; r²=0.360). Our analysis also highlights that an ERFE concentration above a threshold of 100 ng/mL repressed hepcidin more efficiently (data not shown). The elevated concentration of plasma ERFE was associated with a more pronounced degree of ineffective erythropoiesis as assessed by a significant increase of plasma concentration of soluble transferrin receptor (sTfR) in SF3B1^{MUT} compared to SF3B1^{WT} MDS patients (FIG. 3A). Serum EPO level was equally increased in SF3B1^{MUT} and SF3B1^{WT} patients (table 1), and, although ERFE is regulated by erythropoietin in mice, we did not find any correlation between serum EPO level and ERFE concentration suggesting that ERFE production was not restricted to the stimulation of erythroid cells by EPO (FIG. 3B) (27).

To validate these findings, we prospectively enrolled an external cohort of lower risk MDS patients in our study until the number of SF3B1^{MUT} MDS was comparable to that of the learning cohort (table 2). This validation cohort included 55 MDS patients with 42 (78%) of MDS with SF3B1 mutation, and notably, a similar mean number of RBC units/8 weeks between SF3B1^{MUT} and SF3B1^{WT} patients. The mean concentration of ERFE and ferritin were significantly increased (P=0.0005 and P=0.0488, respectively; FIG. 2E, 2F) while hepcidin and hepcidin/ferritin ratio were significantly decreased in SF3B1^{MUT} patients (P=0.0038 and P=0.0002, respectively; FIG. 2G, 2H). This confirms the results of the learning cohort and suggests that the increase of ERFE concentration in SF3B1^{MUT} MDS patients is independent of RBC transfusions.

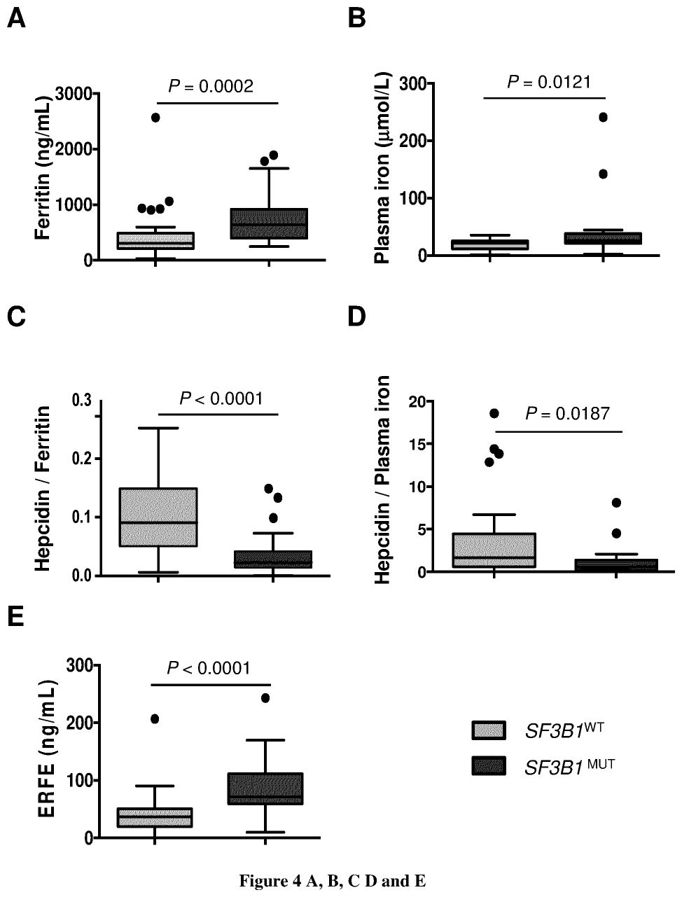

Iron homeostasis changes upon RBC transfusions which diminish the degree of anemia and increase the level of circulating iron, both effects resulting in the up-regulation of hepcidin. To rule out the influence of RBC transfusion in our analysis, we delineated a subset of 61 MDS in the learning cohort including 25 SF3B1^{MUT} and 36 SF3B1^{WT} patients with a low transfusion burden before inclusion (<4 RBC units per 8 weeks). In this subset, patients with SF3B1^{MUT} or SF3B1^{WT} MDS were equally transfused (mean 0.5 RBC unit/8 weeks), but ferritin and plasma irons levels remained higher in SF3B1^{MUT} patients (FIG. 4A, 4B). The hepcidin/ferritin or hepcidin/plasma iron ratios were significantly lower in SF3B1^{MUT} patients (FIG. 4C, 4D) and the circulating ERFE concentration remained significantly more elevated in low transfusion burden SF3B1^{MUT} patients compared to SF3B1^{WT} patients (FIG. 4E).

We then explored the determinants of hyperferritinemia >300 μg/mL in low transfusion burden patients. By univariate analysis, ERFE, hepcidin and SF3B1 mutation, but not sTfR level and the number of transfused RBC units, were significantly linked to the concentration of serum ferritin. By multivariate analysis, ERFE, hepcidin and SF3B1 mutation remained independent predictors of high ferritin level (data not shown). Altogether, these results indicate that, before patients reached a critical threshold of transfusion dependence, hyperferritinemia is caused by SF3B1^{MUT}-induced expression of ERFE, which in turn lowers hepcidin.

Erythroid Lineage-Restricted Expression of ERFE⁺¹²

In mice, ERFE mRNA expression in the BM is regulated by erythropoietin (EPO) and is predominant in basophilic and polychromatic erythroblasts (27). To investigate whether ERFE and ERFE⁺¹² expression is restricted to the erythroid lineage, we amplified in parallel the erythroid or granulocytic precursors deriving from the BM CD34⁺ cells of 1 SF3B1^{MUT} and 1 SF3B1^{WT} sample. The purity of each lineage was assessed by the cytological examination of May-Grünwald-Giemsa-stained cytospins and the quantification of lineage-restricted markers by RT-qPCR (data not shown). To compare the amount of each transcript isoform at the different stages of differentiation, we quantified the canonical ERFE^{WT} and the aberrant ERFE⁺¹² by RT-qPCR (data not shown). The expression of the canonical transcript ERFE^{WT} expressed as Normalized Ratio Quantities (NRQ) increased in both SF3B1^{MUT} and SF3B1^{WT} MDS erythroblasts. In granulocytes, the expression of ERFE^{WT} was close to the detection limit (data not shown). The expression of ERFE⁺¹² was restricted to the erythroid lineage, increased along the differentiation of SF3B1^{MUT} erythroblasts and was higher in SF3B1^{MUT} compared to SF3B1^{WT} erythroblasts (data not shown). These results indicate that ERFE⁺¹² expression is specific to the erythroid lineage.

SF3B1 gene is mutated in 15% of patients suffering from CLL and present in CD19⁺ lymphocytes (14, 30). To investigate whether ERFE⁺¹² was detectable in SF3B1^{uT} CLL, we collected one sample from a patient with a CLL followed by a MDS which BM MNC expressed a clonal SF3B1^{K700E} mutation, and 3 peripheral blood (PB) MNC samples from 2 patients with a CLL, one of them harboring a clonal SF3B1^{T663I} mutation with a variant allele frequency over 40% and the second with no mutation of SF3B1 gene and the third from 1 MDS patient with a SF3B1^{K700E} mutation. The MAP3K7 transcript, already known to be alternatively spliced in SF3B1^{MUT} CLL or MDS by the use of a cryptic 3′ ss, was analyzed (14, 17). A 170 nt fragment corresponding to the alternative MAP3K7 transcript was enriched in all SF3B1^{MUT} samples compared to the SF3B1^{WT} CLL (data not shown). ERFE⁺¹² was detected in the PB MNC of the SF3B1^{K700E} MDS, but not in the SF3B1^{T663I} or SF3B1^{WT} CLL. ERFE⁺¹² was not detected in BM MNC of the SF3B1^{K700E} CLL+MDS sample. To get further insights on the cell types expressing ERFE⁺¹², we sorted myeloid cells containing erythroblasts, CD19⁺CD5⁻ B cells, CD19⁺CD5⁺ pathological B cells and CD3⁺ T cells from the BM MNC fraction of the SF3B1^{K700E} CLL+MDS, one SF3B1^{K700E} MDS and one SF3B1^{WT} MDS, and from the PB MNC of the SF3B1^{T663I} CLL. The number of cells in the myeloid fraction of SF3B1^{T663I} CLL was too small for further studies (data not shown). The 170 nt fragment of MAP3K7 was detected in CD19⁺CD5⁺ pathological B cells of SF3B1^{K700E} CLL+MDS and SF3B1^{T663I} CLL and also in the myeloid fraction, but not in the CD19⁺CD5⁻ B cells of SF3B1^{K700E} MDS (data not shown). By sequencing SF3B1 RNA, we demonstrated that the mutation was present in the cell populations in which the alternative MAP3K7 transcript was detected (data not shown). The alternative ERFE⁺¹² transcript was not detected in the CD19⁺CD5⁺ pathological B cells of SF3B1^{K700E} MDS+CLL or SF3B1^{T663I} CLL and its expression was restricted to SF3B1^{K700E} myeloid MDS cells. Altogether, these results indicate that ERFE is expressed in erythroid cells and ERFE⁺¹² is restricted to SF3B1^{MUT} erythroid cells.

Changes in ERFE⁺¹² Expression Correlated with the Response to Lenalidomide

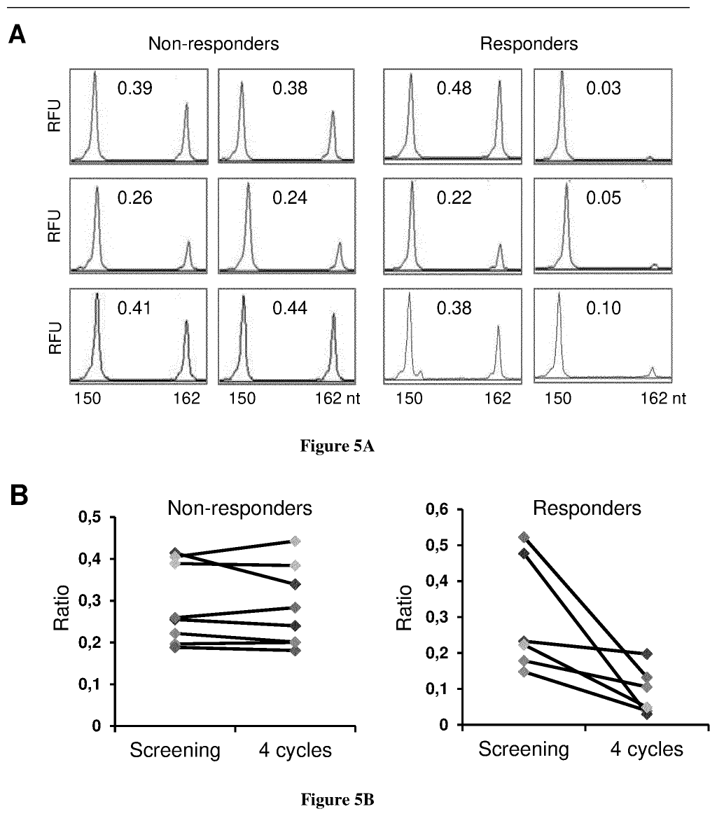

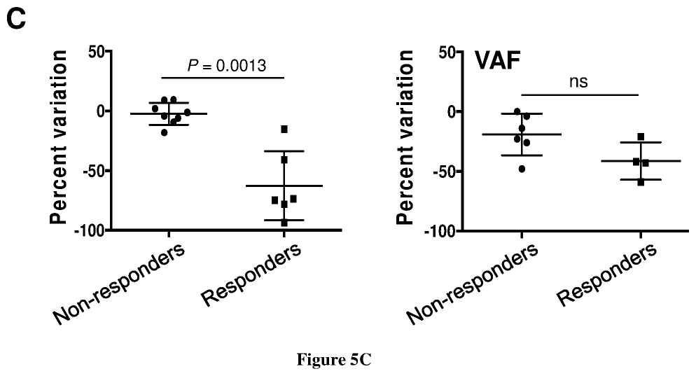

Fifty percent of lower risk MDS patients, including patients with MDS-RS experience primary resistance or secondary failure to treatments they receive to cure their anemia. Whether the mechanism of resistance involves the persistence of clonal erythropoiesis is always unknown. We have previously shown that lenalidomide administered to ESA-resistant non-del (5q) MDS targets the malignant clone and in some cases, eliminate the dominant SF3B1^{MUT} clone for the duration of response (31). However, the frequency of the SF3B1 variant allele, which is expressed in erythroid and myeloid cells does not only reflect the abundance of clonal erythroblasts. Here, we used the expression of erythroid-specific ERFE⁺¹² transcript for the follow-up of patients included in the GFM-LenEpo-08 clinical trial (NCT01718379 at clinicaltrials.gov). For this purpose, we performed fluorescent PCR and integrated ERFE⁺¹² and ERFE^{WT} peak heights as a ratio ERFE⁺¹²/ERFE⁺¹²±ERFE^{WT} in 14 SF3B1^{MUT} MDS patients for whom paired RNA samples were available. The ratio ERFE⁺¹²/ERFE⁺¹²+ERFE^{WT} decreased in 6 responding patients while it remained always stable in 8 non-responding patients (FIG. 5A, 5B). The percent variation of ratios between screening and evaluation at 4 cycles of treatment was significantly different between responding and non-responding patients (Mann-Whitney test P=0.0013; FIG. 5C, left). By contrast, the percent variation of SF3B1 variant allele frequency of BM mononuclear cells between screening and evaluation at 4 cycles was less variable between 6/8 non-responding and 4/6 responding patients (FIG. 5C, right). Altogether, our results demonstrate that ERFE⁺¹² expression allows the evaluation of clonal erythropoiesis under therapy.

### REFERENCES

Throughout this application, various references describe the state of the art to which this invention pertains. The disclosures of these references are hereby incorporated by reference into the present disclosure.
1. D A. Arber, Orazi A, Hasserjian R, Thiele J, Borowitz M J, Le Beau M M, Bloomfield C D, Cazzola M, Vardiman J W. The 2016 revision to the World Health Organization classification of myeloid neoplasms and acute leukemia. Blood.127:2391-405 (2016).
2. V. Chesnais, M.-L. Arcangeli, C. Delette, A. Rousseau, H. Guermouche, C. Lefevre, S. Bondu, M. Diop, M. Cheok, N. Chapuis, L. Legros, S. Raynaud, L. Willems, D. Bouscary, E. Lauret, O. A. Bernard, O. Kosmider, F. Pflumio, M. Fontenay, Architectural and functional heterogeneity of hematopoietic stem/progenitor cells in non-del (5q) myelodysplastic syndromes, Blood 129, 484-496 (2017).
3. P L. Greenberg, Tuechler H, Schanz J, Sanz G, Garcia-Manero G, Sole F, Bennett J M, Bowen D, Fenaux P, Dreyfus F, Kantarjian H, Kuendgen A, Levis A, Malcovati L, Cazzola M, Cermak J, Fonatsch C, Le Beau M M, Slovak M L, Krieger O, Luebbert M, Maciejewski J, Magalhaes S M, Miyazaki Y, Pfeilstocker M, Sekeres M, Sperr W R, Stauder R, Tauro S, Valent P, Vallespi T, van de Loosdrecht A A, Germing U, Haase D. Revised international prognostic scoring system for myelodysplastic syndromes. Blood. 120:2454-65 (2012).
4. V. Santini. Anemia as the Main Manifestation of Myelodysplastic Syndromes. Semin Hematol. 52:348-56 (2015).
5. A. List, Kurtin S, Roe D J, Buresh A, Mahadevan D, Fuchs D, Rimsza L, Heaton R, Knight R, Zeldis J B. Efficacy of lenalidomide in myelodysplastic syndromes. N Engl J Med352:549-57 (2005).
6. A. Toma A, Kosmider O, Chevret S, Delaunay J, Stamatoullas A, Rose C, et al. Lenalidomide with or without erythropoietin in transfusion-dependent erythropoiesis-stimulating agent-refractory lower-risk MDS without 5q deletion. Leukemia. 30:897-905 (2016).
7. V. Chesnais, A. Renneville, A. Toma, J. Lambert, M. Passet, F. Dumont, S. Chevret, J. Lejeune, A. Raimbault, A. Stamatoullas, C. Rose, O. Beyne-Rauzy, J. Delaunay, E. Solary, P. Fenaux, F. Dreyfus, C. Preudhomme, O. Kosmider, M. Fontenay, Effect of lenalidomide treatment on clonal architecture of myelodysplastic syndromes without 5q deletion, Blood 127, 749-760 (2016).
8. R. N. V. S. Suragani, S. M. Cadena, S. M. Cawley, D. Sako, D. Mitchell, R. Li, M. V. Davies, M. J. Alexander, M. Devine, K. S. Loveday, K. W. Underwood, A. V. Grinberg, J. D. Quisel, R. Chopra, R. S. Pearsall, J. Seehra, R. Kumar, Transforming growth factor-β superfamily ligand trap ACE-536 corrects anemia by promoting late-stage erythropoiesis, Nat. Med. 20, 408-414 (2014).
9. M. Dussiot, T. T. Maciel, A. Fricot, C. Chartier, O. Negre, J. Veiga, D. Grapton, E. Paubelle, E. Payen, Y. Beuzard, P. Leboulch, J.-A. Ribeil, J.-B. Arlet, F. Coté, G. Courtois, Y. Z. Ginzburg, T. O. Daniel, R. Chopra, V. Sung, O. Hermine, I. C. Moura, An activin receptor IIA ligand trap corrects ineffective erythropoiesis in β-thalassemia, Nat. Med. 20, 398-407 (2014).
10. R. Tehranchi, R. Invernizzi, A. Grandien, B. Zhivotovsky, B. Fadeel, A.-M. Forsblom, E. Travaglino, J. Samuelsson, R. Hast, L. Nilsson, M. Cazzola, R. Wibom, E. Hellstrom-Lindberg, Aberrant mitochondrial iron distribution and maturation arrest characterize early erythroid precursors in low-risk myelodysplastic syndromes, Blood 106, 247-253 (2005).
11. E. Gyan, E. Frisan, O. Beyne-Rauzy, J.-C. Deschemin, C. Pierre-Eugene, C. Randriamampita, A. Dubart-Kupperschmitt, C. Garrido, F. Dreyfus, P. Mayeux, C. Lacombe, E. Solary, M. Fontenay, Spontaneous and Fas-induced apoptosis of low-grade MDS erythroid precursors involves the endoplasmic reticulum, Leukemia 22, 1864-1873 (2008).
12. Y. Zhu, X. Li, C. Chang, F. Xu, Q. He, J. Guo, Y. Tao, Y. Liu, L. Liu, W. Shi, SF3B1-mutated myelodysplastic syndrome with ring sideroblasts harbors more severe iron overload and corresponding over-erythropoiesis, Leukemia Research 44, 8-16 (2016).
13. L. de Swart, C. Reiniers, T. Bagguley, C. van Marrewijk, D. Bowen, E. Hellstrom-Lindberg, A. Tatic, A. Symeonidis, G. Huls, J. Cermak, A. A. van de Loosdrecht, H. Garelius, D. Culligan, M. Macheta, M. Spanoudakis, P. Panagiotidis, M. Krejci, N. Blijlevens, S. Langemeijer, J. Droste, D. W. Swinkels, A. Smith, T. de Witte, Labile plasma iron levels predict survival in patients with lower-risk myelodysplastic syndromes, Haematologica 103, 69-79 (2018).
14. N. Shenoy, N. Vallumsetla, E. Rachmilewitz, A. Verma, Y. Ginzburg. Impact of iron overload and potential benefit from iron chelation in low-risk myelodysplastic syndrome. Blood. 124: 873-881 (2014).
15. A. F. List, M. R. Baer, D. P. Steensma, A. Raza, J. Esposito, N. Martinez-Lopez, C. Paley, J. Feigert, E. Besa, Deferasirox Reduces Serum Ferritin and Labile Plasma Iron in RBC Transfusion-Dependent Patients With Myelodysplastic Syndrome, JCO 30, 2134-2139 (2012).
16. K. Yoshida, M. Sanada, Y. Shiraishi, D. Nowak, Y. Nagata, R. Yamamoto, Y. Sato, A. Sato-Otsubo, A. Kon, M. Nagasaki, G. Chalkidis, Y. Suzuki, M. Shiosaka, R. Kawahata, T. Yamaguchi, M. Otsu, N. Obara, M. Sakata-Yanagimoto, K. Ishiyama, H. Mori, F. Nolte, W.-K. Hofmann, S. Miyawaki, S. Sugano, C. Haferlach, H. P. Koeffler, L.-Y. Shih, T. Haferlach, S. Chiba, H. Nakauchi, S. Miyano, S. Ogawa, Frequent pathway mutations of splicing machinery in myelodysplasia, Nature 478, 64-69 (2011).
17. E. Papaemmanuil, M. Cazzola, J. Boultwood, L. Malcovati, P. Vyas, D. Bowen, A. Pellagatti, J. S. Wainscoat, E. Hellstrom-Lindberg, C. Gambacorti-Passerini, A. L. Godfrey, I. Rapado, A. Cvejic, R. Rance, C. McGee, P. Ellis, L. J. Mudie, P. J. Stephens, S. McLaren, C. E. Massie, P. S. Tarpey, I. Varela, S. Nik-Zainal, H. R. Davies, A. Shlien, D. Jones, K. Raine, J. Hinton, A. P. Butler, J. W. Teague, E. J. Baxter, J. Score, A. Galli, M. G. Della Porta, E. Travaglino, M. Groves, S. Tauro, N. C. Munshi, K. C. Anderson, A. El-Naggar, A. Fischer, V. Mustonen, A. J. Warren, N. C. P. Cross, A. R. Green, P. A. Futreal, M. R. Stratton, P. J. Campbell, Chronic Myeloid Disorders Working Group of the International Cancer Genome Consortium, Somatic SF3B1 mutation in myelodysplasia with ring sideroblasts, N. Engl. J. Med. 365, 1384-1395 (2011).
18. F. Damm, O. Kosmider, V. Gelsi-Boyer, A. Renneville, N. Carbuccia, C. Hidalgo-Curtis, V. Della Valle, L. Couronné, L. Scourzic, V. Chesnais, A. Guerci-Bresler, B. Slama, O. Beyne-Rauzy, A. Schmidt-Tanguy, A. Stamatoullas-Bastard, F. Dreyfus, T. Prébet, S. de Botton, N. Vey, M. A. Morgan, N. C. P. Cross, C. Preudhomme, D. Birnbaum, O. A. Bernard, M. Fontenay, Groupe Francophone des Myélodysplasies, Mutations affecting mRNA splicing define distinct clinical phenotypes and correlate with patient outcome in myelodysplastic syndromes, Blood 119, 3211-3218 (2012).
19. S. A. Mian, K. Rouault-Pierre, A. E. Smith, T. Seidl, I. Pizzitola, A. Kizilors, A. G. Kulasekararaj, D. Bonnet, G. J. Mufti, SF3B1 mutant MDS-initiating cells may arise from the haematopoietic stem cell compartment, Nature Communications 6, 10004 (2015).
20. T. Mortera-Blanco, M. Dimitriou, P. S. Woll, M. Karimi, E. Elvarsdottir, S. Conte, M. Tobiasson, M. Jansson, I. Douagi, M. Moarii, L. Saft, E. Papaemmanuil, S. E. W. Jacobsen, E. Hellstrom-Lindberg, SF3B1-initiating mutations in MDS-RSs target lymphomyeloid hematopoietic stem cells, Blood 130, 881-890 (2017).
21. R. B. Darman, M. Seiler, A. A. Agrawal, K. H. Lim, S. Peng, D. Aird, S. L. Bailey, E. B. Bhaysar, B. Chan, S. Colla, L. Corson, J. Feala, P. Fekkes, K. Ichikawa, G. F. Keaney, L. Lee, P. Kumar, K. Kunii, C. MacKenzie, M. Matijevic, Y. Mizui, K. Myint, E. S. Park, X. Puyang, A. Selvaraj, M. P. Thomas, J. Tsai, J. Y. Wang, M. Warmuth, H. Yang, P. Zhu, G. Garcia-Manero, R. R. Furman, L. Yu, P. G. Smith, S. Buonamici, Cancer-Associated SF3B1 Hotspot Mutations Induce Cryptic 3′ Splice Site Selection through Use of a Different Branch Point, Cell Rep 13, 1033-1045 (2015).
22. E. A. Obeng, R. J. Chappell, M. Seiler, M. C. Chen, D. R. Campagna, P. J. Schmidt, R. K. Schneider, A. M. Lord, L. Wang, R. G. Gambe, M. E. McConkey, A. M. Ali, A. Raza, L. Yu, S. Buonamici, P. G. Smith, A. Mullally, C. J. Wu, M. D. Fleming, B. L. Ebert, Physiologic Expression of Sf3b1(K700E) Causes Impaired Erythropoiesis, Aberrant Splicing, and Sensitivity to Therapeutic Spliceosome Modulation, Cancer Cell 30, 404-417 (2016).
23. S. Alsafadi, A. Houy, A. Battistella, T. Popova, M. Wassef, E. Henry, F. Tirode, A. Constantinou, S. Piperno-Neumann, S. Roman-Roman, M. Dutertre, M.-H. Stern, Cancer-associated SF3B1 mutations affect alternative splicing by promoting alternative branchpoint usage, Nat Commun 7, 10615 (2016).
24. L. Wang, et al. Transcriptomic Characterization of SF3B1 Mutation Reveals Its Pleiotropic Effects in Chronic Lymphocytic Leukemia, Cancer Cell 30, 750-763 (2016).
25. R. Allikmets, W. H. Raskind, A. Hutchinson, N. D. Schueck, M. Dean, D. M. Koeller, Mutation of a Putative Mitochondrial Iron Transporter Gene (ABC7) in X-Linked Sideroblastic Anemia and Ataxia (XLSA/A), Hum Mol Genet 8, 743-749 (1999).
26. M. Nikpour, C et al. The transporter ABCB7 is a mediator of the phenotype of acquired refractory anemia with ring sideroblasts, Leukemia 27, 889-896 (2013).
27. H. Dolatshad, A. Pellagatti, F. G. Liberante, M. Llorian, E. Repapi, V. Steeples, S. Roy, L. Scifo, R. N. Armstrong, J. Shaw, B. H. Yip, S. Killick, R. Kušec, S. Taylor, K. I. Mills, K. I. Savage, C. W. J. Smith, J. Boultwood, Cryptic splicing events in the iron transporter ABCB7 and other key target genes in SF3B1-mutant myelodysplastic syndromes, Leukemia (2016), doi:10.1038/1eu.2016.149.
28. V. Santini, D. Girelli, A. Sanna, N. Martinelli, L. Duca, N. Campostrini, A. Cortelezzi, M. Corbella, A. Bosi, G. Reda, O. Olivieri, M. D. Cappellini, Hepcidin Levels and Their Determinants in Different Types of Myelodysplastic Syndromes, PLOS ONE 6, e23109 (2011).
29. I. Ambaglio, et al. Inappropriately low hepcidin levels in patients with myelodysplastic syndrome carrying a somatic mutation of SF3B1, Haematologica 98, 420-423 (2013).
30. D. A. Weinstein, C. N. Roy, M. D. Fleming, M. F. Loda, J. I. Wolfsdorf, N. C. Andrews, Inappropriate expression of hepcidin is associated with iron refractory anemia: implications for the anemia of chronic disease, Blood 100, 3776-3781 (2002).
31. G. Nicolas, C. Chauvet, L. Viatte, J. L. Danan, X. Bigard, I. Devaux, C. Beaumont, A. Kahn, S. Vaulont, The gene encoding the iron regulatory peptide hepcidin is regulated by anemia, hypoxia, and inflammation, J Clin Invest 110, 1037-1044 (2002).
32. E. Nemeth, E. V. Valore, M. Territo, G. Schiller, A. Lichtenstein, T. Ganz, Hepcidin, a putative mediator of anemia of inflammation, is a type II acute-phase protein, Blood 101, 2461-2463 (2003).
33. M. Pak, M. A. Lopez, V. Gabayan, T. Ganz, S. Rivera, Suppression of hepcidin during anemia requires erythropoietic activity, Blood 108, 3730-3735 (2006).
34. L. Malcovati, et al. Mieloproliferative, Clinical significance of SF3B1 mutations in myelodysplastic syndromes and myelodysplastic/myeloproliferative neoplasms, Blood 118, 6239-6246 (2011).
35. T. Tanno, N. V. Bhanu, P. A. Oneal, S.-H. Goh, P. Staker, Y. T. Lee, J. W. Moroney, C. H. Reed, N. L. Luban, R.-H. Wang, T. E. Eling, R. Childs, T. Ganz, S. F. Leitman, S. Fucharoen, J. L. Miller, High levels of GDF15 in thalassemia suppress expression of the iron regulatory protein hepcidin, Nature Medicine 13, 1096-1101 (2007).
36. T. Tanno, P et al. Identification of TWSG1 as a second novel erythroid regulator of hepcidin expression in murine and human cells, Blood 114, 181-186 (2009).
37. L. Kautz, G et al. Identification of erythroferrone as an erythroid regulator of iron metabolism, Nature Genetics 46, 678-684 (2014).
38. T. Ganz, et al., Immunoassay for human serum erythroferrone, Blood 130, 1243-1246 (2017).
39. B D. Cheson, et al. Clinical application and proposal for modification of the International Working Group (IWG) response criteria in myelodysplasia. Blood. 108:419-25 (2006).
40. E Hellström-Lindberg, et al. Erythroid response to treatment with G-CSF plus erythropoietin for the anaemia of patients with myelodysplastic syndromes: proposal for a predictive model. Br J Haematol. 99:344-51 (1197).
41. S. Park, et al. GFM group (Groupe Francophone des Myélodysplasies). Predictive factors of response and survival in myelodysplastic syndrome treated with erythropoietin and G-CSF: the GFM experience. Blood. 111:574-82 (2008).

1. A variant of the transcript of ERFE having at least 70% of homology with the nucleic acid sequence SEQ ID NO: 2 or a variant of the protein ERFE having at least 70% of homology with the amino acid sequence SEQ ID NO: 4.

2. A variant of the transcript of ERFE according to claim 1 wherein said variant has a nucleic acid sequence SEQ ID NO: 2 (ERFE⁺¹²).

3. A variant of the protein ERFE according to claim 1 wherein said variant has an amino acid sequence SEQ ID NO: 4 (ERFE^{VPFQ}).

4. A variant of the protein ERFE according to claim 1 wherein said variant comprises at least the amino acids sequence of SEQ ID NO: 7 in its amino acids sequence.

5. A variant of the protein of ERFE according to claim 1 wherein said variant has at least 70% of homology with the SEQ ID NO:3 and comprises in the sequence the 4 amino acids VPFQ of SEQ ID NO: 5.

6. A method for diagnosing an anemia in a patient suffering from a myeloid malignancy comprising,
determining, in a sample obtained from the patient, the expression of a variant of the ERFE transcript or a variant of the ERFE protein of claim 1, wherein the detection of the variant of the ERFE transcript or the variant of the ERFE protein indicates that the patient suffers from an anemia with at least one mutation in the SF3B1 gene.

7. A method which allows to indicate if a treatment of a anemia will or not target the SF3B1-mutated progenitors and/or erythroid precursors in a patient suffering from a myeloid malignancy with at least one mutation in the SF3B1 gene comprising determining, in a sample obtained from the patient, the expression of a variant of the ERFE transcript or a variant of the ERFE protein of claim 1 wherein the detection of such variants indicates that said treatment is effective or not in targeting the clonal/abnormal SF3B1-mutated erythropoiesis.

8. A method of monitoring a treatment of anemia by lenalinomide in a patient suffering from a myeloid malignancy with at least one mutation in SF3B1 gene comprising
determining, in a sample obtained from the patient, i) the expression level of a variant of the ERFE transcript or of a variant of the ERFE protein of claim 1 before and after the treatment by lenalinomide, ii) comparing the expression levels obtained before and after the treatment by lenalinomide, wherein when the expression level of the variants obtained after the treatment by lenalinomide is reduced compared to a the expression level of the variants obtained before the treatment, this indicates that the SF3B1-mutated erythropoiesis is decreased and that the patient responds to the treatment by lenalinomide.

9. A method for diagnosing a systemic iron overload in a patient suffering from a myeloid malignancy with at least one mutation in the SF3B1 gene comprising determining, in a sample obtained from the patient, the expression of a variant of the ERFE transcript or a variant of the ERFE protein of claim 1, wherein the detection of such variants indicates that said patient has a systemic iron overload.

10. A method for predicting a parenchymal iron overload in liver and heart in a patient suffering from a myeloid malignancy with at least one mutation in the SF3B1 gene comprising determining, in a sample obtained from the patient, the expression of a variant of the ERFE transcript or a variant of the ERFE protein of claim 1, wherein the detection of such variants indicates that said patient will have a predisposition to parenchymal iron overload in liver and heart.

11-12. (canceled)

13. An antisense oligonucleotide (ASO) having the following sequences: AACTGAAAGGGAAC (SEQ ID NO: 8), AAAGGGAACCTTGGCAGTGAGGACA (SEQ ID NO: 9) or ACCTTGGCAGTGAGGACATGT (SEQ ID NO: 10).

14-15. (canceled)

16. A method for treating anaemia and/or systemic iron overload in a patient suffering from a myeloid malignancy comprising,
detecting, in a sample obtained from the patient, at least one mutation in an ERFE transcript variant or an ERFE protein variant, and
treating the patient for anaemia and/or systemic iron overload when the at least one mutation is detected.

17. The method of claim 16, wherein the step of treating comprises administering to the patient an inhibitor of the ERFE transcript variant or the ERFE protein variant.

18. The method of claim 17, wherein the inhibitor is an antibody.
